(19) United States
(12) Patent Application Publication
(10) Pub. No.: US 2024/0153607 A1
(43) Pub. Date: May 9, 2024

(54) System for Determining Treatment Timing and Methods of Treatment Timed Based on Biological Process Indicators

(71) Applicant: Arcascope Inc., Arlington, VA (US)

(72) Inventors: Olivia Walch, Falls Church, VA (US); Kevin Hannay, Comfort, TX (US)

(21) Appl. No.: 18/310,791

(22) Filed: May 2, 2023

Related U.S. Application Data
(60) Provisional application No. 63/382,856, filed on Nov. 8, 2022.

(51) Int. Cl. G16H 20/10; G16H 10/60; G16H 40/60; G16H 50/50

(52) U.S. Cl. CPC G16H 20/10; G16H 10/60; G16H 40/60; G16H 50/50

(57) ABSTRACT

A method of treatment and/or a treatment plan for administering a substance to a patient or performing a procedure on the patient can be timed using a modified scheduling process, implemented typically as a computer process, wherein a nonlinear treatment mapping is based on circadian trajectories computed from patient-specific inputs and uncertainty ranges.

### CROSS-REFERENCE

This application claims the benefit of, and priority from, U.S. Provisional Patent Application No. 63/382,856, filed Nov. 8, 2022, entitled, “System for Determining Treatment Timing and Methods of Treatment Timed Based on Biological Process Indicators”.

The entire disclosure(s) of application(s)/patent(s) recited above is(are) hereby incorporated by reference, as if set forth in full in this document, for all intents and purposes.

### FIELD

The present disclosure generally relates to methods of treatment and more particularly to methods of treatment timed based on circadian trajectories computed from biological process indicators.

### BACKGROUND

It is long been known that the time of day a drug is taken can affect its efficacy and/or toxicity. More generally, a biological treatment applied to a human patient or another animal might be done at a particular time of day to maximize efficacy and minimize toxicity. A biological treatment might be the administration of a drug, administration of a nutrient, administration of a behavioral modification, administration of another substance, such as orally, intravenously, intramuscularly, cutaneously, or via other pathways. A biological treatment might be more than, or instead of, administering a substance. For example, a biological treatment might be the application of kidney dialysis, a surgical procedure, or another medical procedure. The term “chronomedicine” and “chronomedical” might be used in the literature to refer to considering time of day effects of biological treatments and “chronomodulation” and “chronomodulated” might refer to scheduling the timing of biological treatments based on time of day and the process might be referred to as “chronotherapy.”

Examples in the literature of chronomedicine and chronomodulation include [Lévi2010], which reported on rodent trials wherein more than forty anticancer drugs were shown to have differing toxicities in depending on the time of administration and/or different efficacy profiles over the course of the day. [Hrushesky] and [Lévi1990] observed that toxicity for cancer treatments in humans can vary by time of day, with doxorubicin and theprubicin found to be most tolerable in the morning, while cisplatin is most tolerable in the afternoon. [Lévi1997] reported up to a five-fold reduction in grade 3-4 mucositis and half the occurrence of neuropathy when drugs are given in a chronomodulated way. More recently, time-of-day effects for overall treatment efficacy have been reported for temozolomide, a glioblastoma treatment [Damato], and immunotherapy [Qian], with morning dosing superior for both. Cancer, while the disease state with the most chronomedical research behind it, is far from the only one: morning versus evening effects have also been reported for treatments for conditions such as asthma, diabetes, and hypertension.

[Sato] describes how local and systemic metabolic responses to exercise can vary based on time of day.

Current methods of chronomedicine and chronomodulation are quite limited and improvements are needed.

### REFERENCES

[Adam] Adam, “Core Concepts: Emerging Science of Chronotherapy Offers Big Opportunities to Optimize Drug Delivery,” (2019) PNAS 116(44):21957-59 [doi:org/10.1073/pnas.1916118116].
[Damato] Damato et al., “A Randomized Feasibility Study Evaluating Temozolomide Circadian Medicine in Patients with Glioma,” (31 Jan. 2022) Neuro-Oncology Practice 9(3):193-200 [doi: 10.1093/nop/npac003].
[Damato2021] Damato et al., “Temozolomide Chronotherapy in Patients with Glioblastoma: A Retrospective Single-Institute Study,” (2 Mar. 2021) Neuro-Oncology Advances 3(1):1-11, vdab041 [doi:org/10.1093/noajnl/vdab041].
[Giacchetti] Giacchetti et al. “Sex Moderates Circadian Chemotherapy Effects on Survival of Patients with Metastatic Colorectal Cancer: A Meta-Analysis,” (2012) Annals of Oncology 23(12):3110-3116 [doi:org/10.1093/annonc/mds148].
[Hill] Hill et al., “Optimizing Circadian Drug Infusion Schedules towards Personalized Cancer Chronotherapy,” (1985) PLoS Computational Biology 16(1):e1007218 (2020) [doi:org/10.1371/journal.pcbi.1007218].
[Hrushesky] Hrushesky, “Circadian Timing of Cancer Chemotherapy.” (1985) Science 228(4695):73-75 [doi: 10.1126/science.3883493].
[Kramer] Kramer et al., “Foundations of Circadian Medicine,” (24 Mar. 2022) PLoS Biology 20(3):e3001567 [doi: 10.1371/journal.pbio.3001567; PMID: 35324893; PMCID: PMC8946668].
[Lee] Lee et al., “Time-of-Day Specificity of Anticancer Drugs May be Mediated by Circadian Regulation of the Cell Cycle,” (12 Feb. 2021) Science Advances 7(7): eabd2645 [doi: 10.1126/sciadv.abd2645].
[Lévi1992] Lévi et al., “A Chronopharmacologic Phase II Clinical Trial with 5-Fluorouracil, Folinic Acid, and Oxaliplatin Using an Ambulatory Multichannel Programmable Pump, High Antitumor Effectiveness against Metastatic Colorectal Cancer,” (1992) Cancer 69(4):893-900 [doi: 10.1002/1097-0142(19920215)69:4<893::AID-CNCR2820690410>3.0.CO; 2-X].
[Lévi1997] Lévi et al, “Randomised Multicentre Trial of Chronotherapy with Oxaliplatin, Fluorouracil, and Folinic Acid in Metastatic Colorectal Cancer,” (1997) The Lancet 350(9079):681-86 [doi:org/10.1016/S0140-6736(97)03358-8].
[Lévi2010] Lévi et al., “Circadian Timing in Cancer Treatments,” (2010) Annual Review of Pharmacology and Toxicology 50:377-421 [doi.org/10.1146/annurev.pharmtox.48.113006.094626].
[Lévi1990] Lévi, “Meeting Report: Chronotherapy of Cancers: A New Strategy in Clinical Oncology,” (1990) Chronobiology International 7(1):81-84 [doi.org/10.3109/07420529009056957].
[Montaigne] Montaigne et al., “Daytime Variation of Perioperative Myocardial Injury in Cardiac Surgery and Its Prevention by Rev-Erba Antagonism: A Single-Centre Propensity-Matched Cohort Study and a Randomised Study,” (2018) The Lancet 391(10115):59-69 [doi:org/10.1016/S0140-6736(17)32132-3].
[Mormont] Mormont et al., “Cancer Chronotherapy: Principles, Applications, and Perspectives,” (2003) Cancer 97(1):155-169 [doi:org/10.1002/cncr.11040].
[Münch] Münch et al., “Timing Matters: New Tools for Personalized Chronomedicine and Circadian Health,” (2019) Acta Physiologica 227(2):e13300 [doi:org/10.1016/S0140-6736(17)32132-3].
[Peeples] Peeples, “Medicine's Secret Ingredient—it's in the timing” (2018) Nature News Feature 556(7700):290-292 [doi.org/10.1038/d41586-018-04600-8].
[Qian] Qian et al., “Effect of Immunotherapy Time-of-Day Infusion on Overall Survival among Patients with Advanced Melanoma in the USA (MEMOIR): A Propensity Score-Matched Analysis of a Single-Centre, Longitudinal Study,” (12 Nov. 2021) The Lancet Oncology 22(12):1777-1786 [doi:org/10.1016/S1470-2045(21)00546-5].
[Sato] Sato et al., “Atlas of Exercise Metabolism Reveals Time-Dependent Signatures of Metabolic Homeostasis,” (1 Feb. 2022) Cell Metabolism, 34(2):329-345, e1-e8 [doi:org/10.1016/j.cmet.2021.12.016].
[Selfridge] Selfridge et al., “Chronotherapy: Intuitive, Sound, Founded . . . But Not Broadly Applied,” (2016) Drugs 76:507-1521 [doi:org/10.1007/s40265-016-0646-4].
[Wang] Wang et al., “The Circadian Immune System,” (3 Jun. 2022) Science Immunology 7(72):eabm2465 [doi: 10.1126/sciimmunol.abm2465].
[Wang] Wang et al., “Time of Day of Vaccination Affects SARS-CoV-2 Antibody Responses in an Observational Study of Health Care Workers,” (1 Feb. 2022) Journal of Biological Rhythms 37(1):124-129 [doi:org/10.1177/07487304211059315].
[Zhang] Zhang et al., “A Circadian Gene Expression Atlas in Mammals: Implications for Biology and Medicine,” (2014) PNAS 111(45):16219-16224 [doi:org/10.1073/pnas.1408886111].

### SUMMARY

A method of treatment and/or a treatment plan for administering a substance to a patient or performing a procedure on the patient can be timed using a modified scheduling process, implemented typically as a computer process, wherein a nonlinear treatment mapping is based on circadian trajectories computed from patient-specific inputs and uncertainty ranges.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of methods and apparatus, as defined in the claims, is provided in the following written description of various embodiments of the disclosure and illustrated in the accompanying drawings.

### BRIEF DESCRIPTION OF THE DRAWINGS

Various embodiments in accordance with the present disclosure will be described with reference to the drawings, in which:

FIG. 1 is an illustration of a trajectory.

FIG. 2 is an illustration of a circadian trajectory.

FIG. 3 is an illustration of a trajectory bundle.

FIG. 4 is an illustration of a circadian trajectory bundle.

FIG. 5 illustrates a set of trajectories for clock gene expression given a light exposure history.

FIG. 6 illustrates trajectory bundles, one for light histories, such as those shown in FIG. 5, and one for clock gene expression circadian trajectories, such as those shown in FIG. 5.

FIG. 7 illustrates an example of a trajectory having some uncertainty built in.

FIG. 8 illustrates an example circadian-mapping profile, an efficacy profile, that maps between circadian states to efficacy of a drug.

FIG. 9 illustrates another example circadian-mapping profile, a toxicity profile, that maps between circadian states to toxicity of a drug.

FIG. 10 is a diagram of a patient treatment system, according to various embodiments.

FIG. 11 is a diagram of a treatment assignment and coordination system, according to various embodiments.

FIG. 12 is a diagram of a treatment scheduling feedback system, according to various embodiments.

FIG. 13 illustrates components that might be used in a control system to change environmental cues and behavioral triggers in response a fixed treatment time and user data, according to various embodiments.

FIG. 14 illustrates components that might be used for a digital twin simulation, according to various embodiments.

FIG. 15 illustrates components that might be used for a profile identification system, according to various embodiments.

FIG. 16 illustrates components that might be used for a treatment mapping module, according to various embodiments.

FIG. 17 illustrates components that might be used for a treatment system module, according to various embodiments.

FIG. 18 illustrates components that might be used for a control system, according to various embodiments.

FIG. 19 illustrates components that might be used for a treatment assignment and coordination system, according to various embodiments.

FIG. 20 illustrates another example of a trajectory uncertainty, according to various embodiments.

FIG. 21 illustrates an example computer system memory structure as might be used in performing methods described herein, according to various embodiments.

FIG. 22 is a block diagram illustrating an example computer system upon which the systems illustrated in FIGS. 1 and 21 may be implemented, according to various embodiments.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

In a patient treatment system, various inputs are obtained, such as wearable device data from a wearable device worn by a patient, other sensor data from the patient, calendar data from a calendar program of the patient, location, travel, environmental, etc. inputs. From some or all of those inputs, as explained herein, and possibly also from precomputed machine-learning models and stored data that is not necessarily specific to that patient, a circadian trajectory that maps between wall-clock time and a circadian state is determined. The circadian trajectory might have varying degrees of uncertainty from patient to patient and/or from time to time for a given patient, such as when fewer inputs are available, inputs are inconsistent, and/or for other reasons. The patient treatment system can determine, from the circadian trajectory and circadian-mapping profiles for a given treatment, what wall-clock time, times, and/or time ranges, to administer a treatment.

A highly accurate measurement of a circadian state of a patient at given point in time can be obtained with great inconvenience to the patient and a considerable delay in processing. For example, multiple saliva samples can be taken while the patient resides in a dark room for many hours but that is typically impractical and the circadian states and the circadian trajectory of the patient over those hours might only be known after lab results are complete, perhaps days later and therefore of limited use. Using methods and apparatus described herein, a good estimate of the circadian state can be obtained in near real-time with minimal effort and inconvenience to the patient. In some instances, the methods and apparatus described herein might not be able to determine the circadian state with great accuracy at some times (such as when the patient is involved in travel or irregular activities) while being able to be more accurate at other times.

The time, or time ranges, for administering a treatment or signaling for the administration to occur, might be determined based on circadian-mapping profiles that indicate, for example, what circadian states relate to high efficacy and/or low toxicity. In a specific example, if a patient's circadian state at a snapshot in time is represented in memory by a value between 0.0 and 1.0 and the patient treatment system determines that efficacy for a drug D is highest at a state value of CS=0.4 and toxicity of drug D is lowest at that state value as well, and further that the patient treatment system determines that CS=0.4 corresponds to 10 AM with an uncertainty of three minutes, the patient treatment system might send a message to the patient, perhaps on a wearable device and perhaps some time in advance, to the effect of “Please take your pill D sometime between 9:57 and 10:03 AM for best results.”

Other scales are possible for circadian state, such as circadian state being considered like a circular phase and continuously varying from 0 through to 2*π (units of radians), which the patient treatment system could consider to be the same as 0. A circadian trajectory can represent past circadian state changes as a function of wall-clock time and/or anticipated or predicted future circadian state changes as a function of wall-clock time. Wall-clock time could be represented by a clock circuit, a computer circuit that keeps time, and/or a computer element that receives signals representing a time measured independent of bodily activity or details such as a local time, which might have varying resolutions, such as from {morning, daytime, noon, afternoon, evening, late night}, to HH o'clock, to HH:MM on day DD, to HH:MM:SS on day DD of month M in year Y.

Wall-clock time can be represented in a number of ways, such as a stored value of between 0.0 and 1.0 representing a time of day, perhaps in resolutions of seconds or some other interval, a stored value of between 0:00 and 23:59:59 representing a time of day, an epoch time, such as a number of elapsed seconds since some specified time and date. As an example of epoch time, a stored time value, t, where t=1642724939 might represent a wall-clock time of 12:28:59 AM UTC on Jan. 21, 2022.

In some instances, a patient treatment system might maintain multiple circadian trajectories for a patient and use one or more of those maintained circadian trajectories for particular treatment management. Examples of multiple maintained circadian trajectories might include a central circadian trajectory that maps a body's central circadian state to wall-clock time where the central circadian state corresponds to the body's suprachiasmatic nucleus (SCN), such as a representation in memory of instantaneous firing patterns in the suprachiasmatic nucleus, corresponding to how neuronal firing patterns change over time, and/or patterns in changes of concentration of clock genes within cells.

A peripheral circadian state might reflect circadian-relevant molecular concentrations in a body and a peripheral circadian trajectory representing changes in that peripheral circadian state over a period of wall-clock time. For example, one particular peripheral circadian state might represent a current concentration of sodium-proton exchanger (NHE3) in the intestinal lumen of a patient.

Examples of treatments might include administering a medicine to the patient, outputting a message to be received by the patient for them to take the medicine, and/or indicating an optimal time, or estimate thereof, for some other treatment. Where the treatment has a fixed wall-clock time, such as a surgery scheduled for November 18 at 2:00 PM, the patient treatment system could compute which inputs could affect the circadian trajectory and provide the patient with inputs, and/or administer some pre-surgery treatment, that would be expected to cause a shift in the patient's circadian trajectory such that a desired circadian state aligns with the fixed wall-clock time of the treatment.

Determining the circadian trajectory can be an involved process, since some input data might be missing and/or there might be some disruptions in the patient's normal schedule, such as travel over several time zones. In some cases, a circadian trajectory might need to be computed on the fly, in real time, or near real time and therefore some optimizations in the processing of data might be required. Furthermore, some of the computations might be done on a low-powered device. In some implementations, part of the computation is done remotely, perhaps on more powerful servers, and part of the computation is done on a low-power, local, wearable and/or portable device with communications capability.

In a typical configuration, a circadian trajectory is continuous in that two nearby points on a circadian trajectory map to nearby wall-clock times. While a circadian trajectory could map exactly to wall-clock time, e.g., a linear mapping, more typically a circadian trajectory will compress or expand units of circadian time relative to wall-clock time, which might be treated as a circadian clock and a wall clock running at different rates and the ratio of the different rates varying over a day or other measurement period. In general, a complete circadian trajectory can provide a mapping from a circadian state to a wall-clock time as well as providing a mapping from a wall-clock time to a circadian state. A circadian trajectory is also recurrent in nature with the previous estimate being fed into the prediction over the next timestep. Circadian trajectories also produce estimates on a time scale significantly shorter than the period of the oscillator (approximately 24 hours). For example, the circadian trajectory could estimate the state of the oscillator every 6 minutes, producing 240 predicted or forecasted states for each day.

Whereas some treatments might be prescribed strictly on wall-clock times (e.g., “take one pill early in the morning and a second one at noon local time”), other treatments might rely on circadian time that represents some circadian state on a circadian trajectory. Such other treatments might benefit a patient more if that circadian trajectory and/or a current circadian state is more accurately and/or precisely determined.

The circadian trajectory can be represented and stored in computer memory as a lookup-table, possibly corresponding to a piecewise linear plot of circadian time versus wall-clock time. The patient treatment system might store the circadian trajectory as a vector data structure that can be manipulated by computer instructions created for performing vector operations or hardware capable of vector operations. The resolution of such a plot of circadian time versus wall-clock time might be on the order of seconds or minutes. For example, a 24-hour plot of circadian time versus wall-clock time might be stored as a vector of 288 values each corresponding to a circadian state at five-minute intervals.

A patient's body has biological states that might vary over time. For example, a stored biological state value might reflect a concentration of melatonin in the patient's saliva, the concentration of caffeine in the patient's bloodstream, the concentration of the Bmall gene in the patient's brain, the current firing rate in the patient's suprachiasmatic nucleus, etc. For nonhuman patients, some biological states might not have human counterparts. A circadian state is one particular example of a biological state.

A patient's circadian state is a biological state corresponding to where the patient is in a circadian cycle. As explained herein, a given body might have multiple circadian cycles, not all of which need be aligned, such as a central circadian cycle and peripheral circadian cycles. Melatonin concentrations, Bmall gene concentrations, firing rate of the SCN, and other biological states might be circadian states or proxies therefore.

A time sequence of a biological state can be stored and/or represented in memory as a time series of state values, or a trajectory for the biological state. The representation of a trajectory in computer memory can be in the form of a listing of records each indicating a state value and a time, a piecewise linear plot, coefficients of a fitted curve, or other form of data structure usable for representing state values and specified times.

An example biological state might be that a melatonin concentration is 4.3 picograms/ml at epoch time t=1642724939. Another example biological state might be that core body temperature is 97.3° F. at epoch time t=1642724939. A value, R, might be stored in memory as a representation of some unitless or unit-specific quantity corresponding to a biological state. For example, it might be a value of a biological oscillator such as melatonin concentration in saliva in units picogram per milliliter. Another example is a unitless quantity corresponding to a measure of cohesion of neurons firing in a suprachiasmatic nucleus and might be such that R=0 represents a state wherein the neurons are completely out of sync with each other and R=1 represents a state wherein the neurons are firing perfectly in sync with each other.

As an example of a circadian state, it might be represented as a sinusoidal curve defined by having an amplitude of Rₐ=0.4 and at an epoch time of t=1642724939 having a phase of ΨP=π/4. A period of a trajectory, which might correspond with a circadian cycle might not be constant and can change from period to period (e.g., a 24.75 hour circadian cycle followed by a 25.4 hour circadian cycle then followed by a 24.9 hour circadian cycle. The effective period of a trajectory, the rate of change of the state might also change dynamically over the course of a trajectory as time shrinks and expands.

In some cases, an intrinsic period might be present and/or measured, which might correspond to a circadian cycle that a body experiences in the absence external time-marking signals, such as light variations throughout the day. A patient treatment system might store a value for the intrinsic period that can be used as a parameter that can be tuned to improve estimates of a person's circadian state, along with other parameters that might really matter to the circadian phenotype, like light sensitivity, in defining a trajectory. Varying such parameters can be used to generate a trajectory bundle, and the range of parameters can be narrowed or made wider if other information about them is available, such as by giving a person a test to see how much their pupil constricts to assess light sensitivity, or demographic correlations are discovered which impart information about the parameters under consideration.

In another example, a circadian state might be stored as a representation of multiple gene expressions, such as a vector value [32.242, 484.23, 4994.2 . . . ] for t=16427249 representing expression levels for genes [Gene₁, Gene₂, Gene₃, . . . ]. For some such circadian states, there might be hundreds of genes represented.

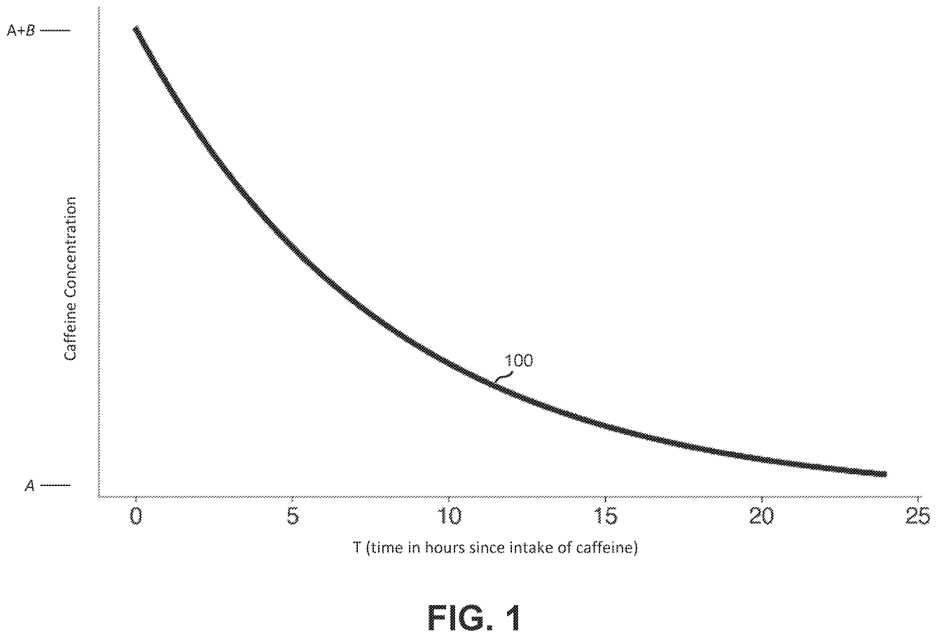

FIG. 1 is an illustration of a trajectory 100. In this example, the biological state is a body's caffeine concentration over time following the intake of caffeine at time t=0. In the plot shown, the scale of caffeine concentration ranges from A (which can be 0, or not) through A+B, which can be an arbitrary scale or a stored trajectory could include specific scales. In this particular example, the caffeine concentration appears to decline with exponential decay. For some trajectories, the biological state might be measured at a few points in time and then fitted to a curve that assumes certain behaviors, such as linear or exponential decay.

A trajectory maps between wall-clock time and a biological state, as a time series, either in the past or future, of biologically relevant data. An example might be a trajectory of adenosine concentrations over time, a trajectory of TMZ concentration over time, etc. A trajectory might reflect a half-life of a drug concentration in the body, interactions between drugs and other molecules in the body over time, etc.

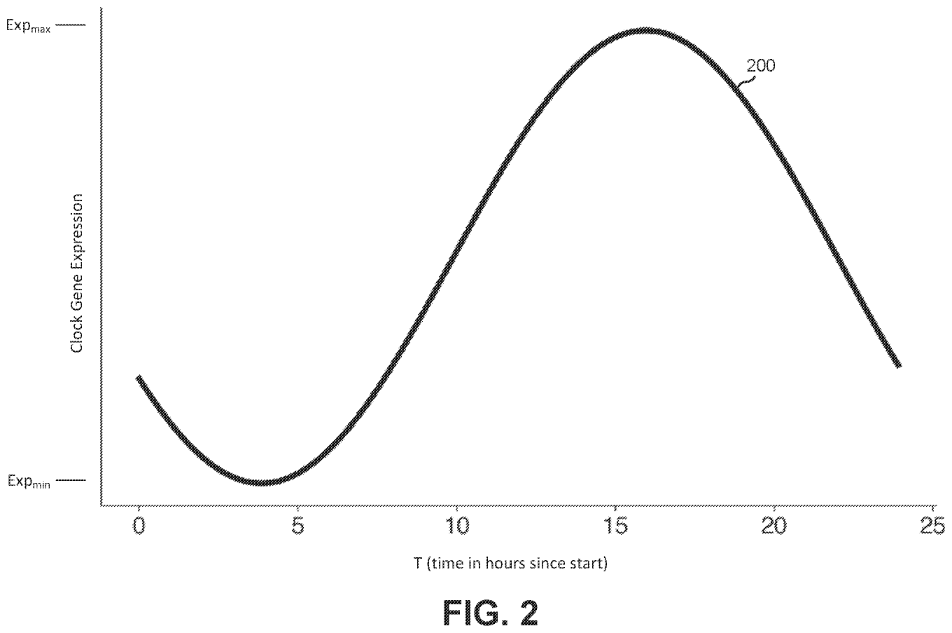

FIG. 2 is an illustration of a circadian trajectory 200, which represents a time series of circadian states. An example might be a biological state representing changes in melatonin in saliva over the course of a day, levels of firing patterns in the brain over the course of weeks, or expression rates of clock genes over either time scale. While the example of circadian trajectory 200 suggests a sinusoidal pattern with a period of around twenty-four hours, that need not be the case for all circadian trajectories.

In addition to storing and manipulating a trajectory of a biological state, a patient treatment system might store, manipulate, and/or operate on a bundle of multiple related trajectories. Different trajectories in a trajectory bundle might be created with different starting conditions and/or assumptions. A trajectory bundle may comprise a sampling of circadian trajectories, which should be considered a finite representation taken from a continuous trajectory probability distribution function (TPDF) or may encapsulate a representation of the full distribution function (TPDF) or an approximation of this function. Therefore, bundles may also be represented or obtained by the expression of the TPDF and the dynamics may be described by the evolution of any representation of this function.

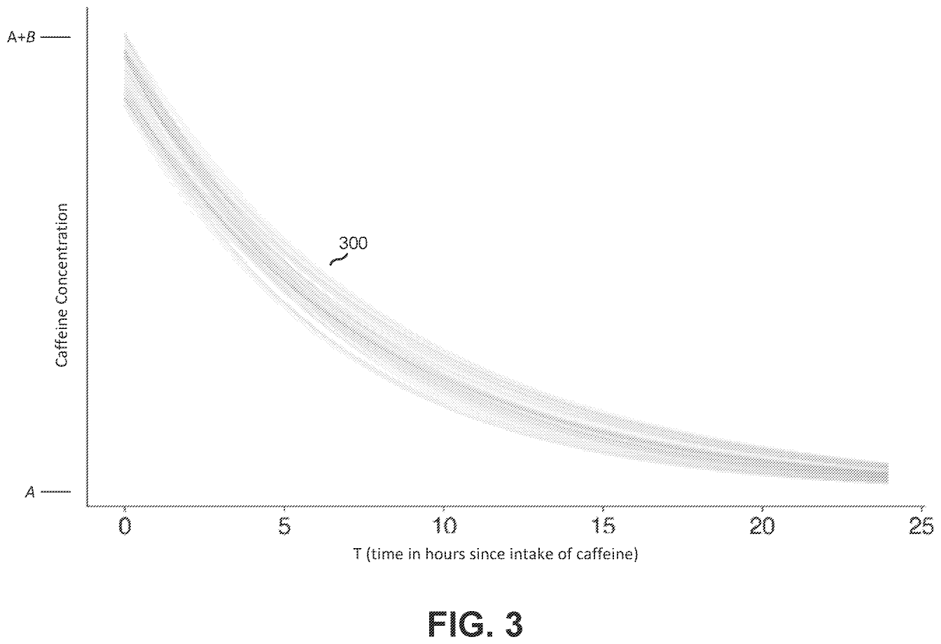

FIG. 3 is an illustration of a trajectory bundle 300 of trajectories representing caffeine concentration in a body. The different trajectories might represent different individuals, different measurements from one person (or multiple people), and/or one set of measurements with differing assumptions or initial conditions applied. For example, the value A+B shown in the plot in FIG. 3 could be a normalized starting concentration for different amounts of caffeine intake to illustrate the probability of various biological states (“fuzziness”) of the trajectory over multiple samples and over multiple caffeine intake amounts. In other examples of trajectory bundles, a trajectory bundle might represent trajectories for differing average concentrations of adenosine receptors in the brain. For example, a trajectory bundle for four different coffee sizes that were ingested by a patient might be stored as a trajectory bundle of four trajectories, while a trajectory bundle for adenosine receptors might contain a sampling of ten trajectories, and a trajectory bundle of one trajectory for each combination of ingestion size and receptor concentration might contain forty trajectories.

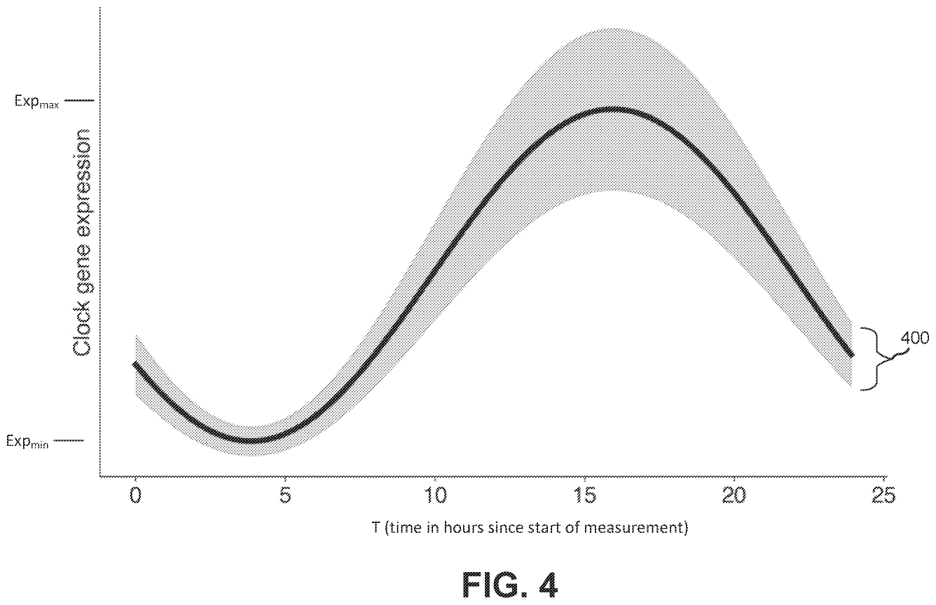

FIG. 4 is an illustration of a circadian trajectory bundle 400. In this example, each trajectory corresponds to a clock gene expression level ranging from Expₘᵢₙ to Expₘₐₓ. For each trajectory, an expression level at time t=0 is the same or is normalized to be the same, as illustrated but that is not necessarily required for circadian trajectory bundles.

A trajectory bundle can be used to quantify uncertainty in a wearable signal. A digital twin simulation can be carried out for 100,000 slight modifications of a wearable history, where each wearable history corresponds to a different trajectory. The spread of biological states shown across the trajectory bundle at every time point can provide the uncertainty for that time point.

A trajectory bundle can be used to quantify uncertainty in a user's circadian phenotype, such as their light sensitivity or intrinsic Tau. A digital twin simulation can be carried out with 10,000 choices of circadian parameter Tau, where each choice of Tau corresponds to a different trajectory. The spread of trajectories at every time point can again provide the uncertainty for that time point. The uncertainty can be used to set the width of the administration window. A high uncertainty will correspond to a wide administration window, while low uncertainty corresponds to a narrow administration window.

A trajectory bundle can be used to patch missing data. In the absence of wearable data, for instance, a digital twin simulation can be carried out over a distribution of possible wearable data histories. These wearable data histories can be chosen from a uniform distribution or one informed by historical data from the user (e.g., “typical days”). The trajectory bundle can patch the gap during the missing data period, reducing the uncertainty in the circadian trajectory when the wearable data resumes. For instance, the circadian state at the moment the wearable data returns can be taken from the average state of the trajectory bundle, and the uncertainty at that moment can also be taken from the trajectory bundle.

A trajectory bundle can be used to quantify the risk of drug interactions. For instance, a trajectory bundle can be used to calculate the risk of two drugs interacting, where the different trajectories include different effective half-lives of the drugs in the system. The risk of drug interactions, calculated from a trajectory bundle, can be used to inform the treatment mapping. An administration window may not be recommended if the risk of drug interactions is too high.

Trajectory bundles can be used to speed up the work of an expensive trajectory mapping. Optimization operations can often become prohibitively slow as complexity increases. For instance, a trajectory bundle can be calculated for the next two days using probabilistic wearable histories (“typical days”). The treatment mapping can then be applied to this hypothetical data in advance of a user request for an administration window. The treatment mapping will yield administration windows for each trajectory in the bundle. When the user next asks for an administration window, the trajectory most closely resembling their actual trajectory will be identified and the administration window corresponding to it will be presented.

An administration window is a dosage amount or treatment with an affiliated period of time recommended for the dose or treatment, calculated from a circadian trajectory, where states might have affiliated uncertainties, and one or more circadian-mapping profiles, such as efficacy profiles and/or toxicity profiles. A patient treatment system might generate an administration window by taking into account other biological trajectories as inputs, such as a trajectory of caffeine leaving the body. An administration window might be represented by a data structure that indicates a timing window and a dosage amount or other treatment suggestions and/or details.

An example of an administration window might be data indicating an administration window for M mg of drug D between 2:00 and 2:30 PM based on the patient treatment system determining that a computation of the expected circadian state in that time period would have low uncertainty and the expected circadian state maps, via a circadian-mapping profile, to a high efficacy and/or low toxicity for drug D. Another example might be data indicating an administration window for M mg of drug D sometime in the afternoon based on the patient treatment system determining that a computation of the expected circadian state in that time period would have low uncertainty and the expected circadian state maps, via a circadian-mapping profile, to a high efficacy and/or low toxicity for drug D where the expected circadian state has a larger uncertainty.

The patient treatment system might provide messages to the patient that reflect the uncertainty. For example: (a) “Take one pill of TMZ between 8:00 AM and 8:15 AM tomorrow” (low uncertainty), (b) “Take 45 mg of your drug tomorrow afternoon” (high uncertainty), and (c) “Take your pill tomorrow” (extremely high uncertainty).

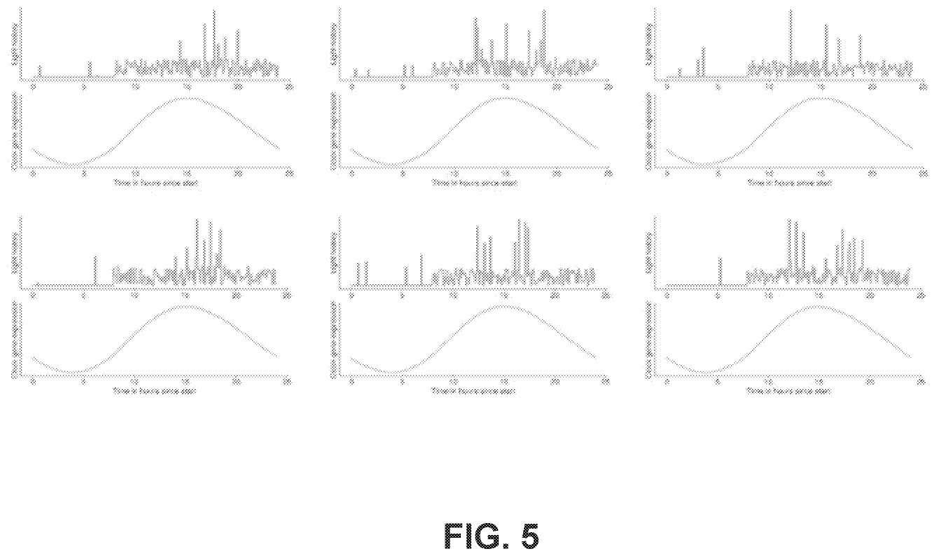

FIG. 5 illustrates a set of trajectories for clock gene expression given a light exposure history. The light exposure histories might be obtained from a wearable device that periodically measures the amount of light or characteristics of light that the wearer is experiencing. The corresponding clock gene expression levels illustrated in FIG. 5 might be determined from actual measurements of the wearer or might be determined or estimated based on a precomputed volatile or mapping from light histories to clock gene expressions.

As shown there in FIG. 5, each of the six examples shows light history represented by a plot of light intensity over time (around 24-25 hours), which could be on some linear or nonlinear scale with higher intensity (e.g., more light) being higher and low light intensity (or no light) being nearer the X-axis in the plots. The corresponding clock gene expression plot over that same time span might be scaled based on a measured or predetermined minimum and maximum clock gene expression values. While not always the case, in the examples shown in FIG. 5, generally periods with more light exposure correspond with higher clock gene expression. It should be understood that while the clock gene expression curves appear generally sinusoidal, that need not be the case.

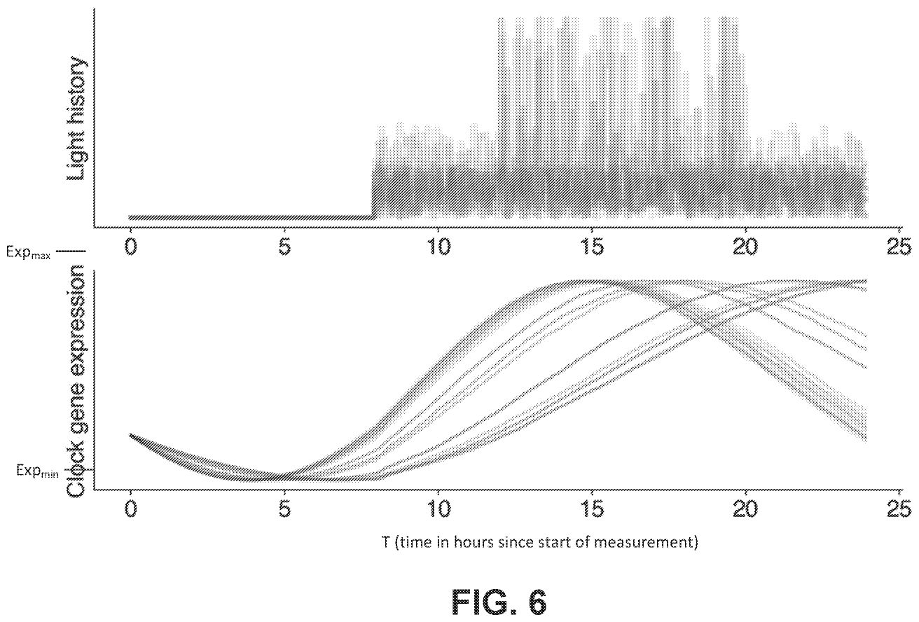

FIG. 6 illustrates trajectory bundles, one for light histories, such as those shown in FIG. 5, and one for clock gene expression circadian trajectories, such as those shown in FIG. 5. A trajectory bundle might bundle 10,000 trajectories, each representing a patient being exposed to 10,000 distinct constant light levels over a one-hour period, or some other period. These can be from actual exposures that are measured or from a model of such exposures. A circadian trajectory bundle might reflect circadian trajectories that would occur for patients with fifty differing levels of light sensitivity, for fifty trajectories. A circadian trajectory bundle might include a circadian trajectory for each combination of the 50 different levels of light sensitivity and 10,000 light levels, for a total of 500,000 circadian trajectories within the circadian trajectory bundle.

A trajectory bundle can comprise circadian trajectories or other biological trajectories. In the presence of missing data, a trajectory bundle can be created by propagating forward from a last tracked circadian state and its affiliated uncertainty with inputs drawn from a distribution. The distributions can be uniform (e.g., sample uniformly across all possible inputs) or informed by historical data (e.g., sample from the space of “typical” days for this person, or sample from the space of likely chronotypes). The inputs can be wearable data inputs such as a hypothetical lighting or activity history. The inputs can be parameters capturing the way the circadian system works or is expected to work, such as greater light sensitivity or reduced light sensitivity affecting state. These can be averaged together to yield the most likely trajectory during the period of missing data.

In the presence of an expensive optimization problem, such as the case of multiple interacting drugs and inputs, trajectory bundles can be precomputed to save time for a patient. For example, if a treatment decision is to be made or a message communicated to a patient and that needs to be done relatively quickly after the patient has made a request (e.g., the patient presses a button on their wearable device and expects a message shortly thereafter), trajectory bundles can be precomputed with probabilistic future paths. Expensive optimization problems can thus be carried out over the set of trajectories ahead of time so that the moment the patient makes a request, the data can be accessed. An updated trajectory can be compared against precomputed trajectories, and if one matches sufficiently well, that one is used. Otherwise, a relatively computationally expensive optimization might take place.

Additionally, the trajectory bundle could be expressed using the full trajectory density function or equivalently coordinates for this density function which can be used to recover the probability density function. For example, through using the observable coordinates defined through Koopman operator theory, the moments of the probability distribution, Fourier coefficients, etc. The optimization problem under consideration may be more efficiently and/or accurately solved when framed under these coordinate transformations.

The patient treatment system might use digital twin modeling for generating trajectories or presumed trajectories from input data. With digital twin modeling, the patient treatment system operates on a virtual model of the patient given some inputs. The inputs might be from body sensors, wearable devices, internal measurements, etc. that might have varying degrees of accuracy and/or precision and some inputs might have time gaps or missing data. Input data can also be physiological parameters, such as light sensitivity. Input data can also be demographics data, like age. The digital twin modeling might include some simulations run to estimate what the actual trajectories might be under certain conditions. The input data can be prerecorded or past obtained data, or might be real-time data. Digital twin modeling might also involve feedback of simulation results. Examples of hardware that might be used for digital twin modeling is described herein elsewhere.

A particular model for generating an optimal time for taking a drug by associating a circadian time with a time for taking a drug might include a raw model output with a model of a core circadian pacemaker connected to another model, such as a blood-brain barrier model and/or a liver model.

State values that result in a trajectory can have affiliated uncertainties thus making the trajectory have some uncertainty. Uncertainty might relate to a confidence level that a state at a given time is correct. High uncertainty might result when the data used to arrive at the state—for instance, wearable data used to predict circadian states—is sparse or highly irregular. Efficacy and toxicity profiles may look very different. The efficacy and/or toxicity profile identification system may identify efficacy profiles with very different peaks from toxicity profiles.

A computer-implemented method for administering a treatment can be provided, perhaps under the control of one or more computer systems configured with executable instructions, for determining a set of patient inputs, determining a treatment, estimating a circadian trajectory of the patient, determining one or more circadian-mapping profiles, determining, from the circadian trajectory and the one or more circadian-mapping profiles, a preferred treatment time period, and then administering the treatment in response to an alert that the preferred treatment time period is occurring or is to occur. The periods and trajectories can vary. The treatment can be an administration of a substance, wherein the substance is one or more of a drug, nutrient, or medicament. The set of patient inputs can comprise data derived from signals received of a patient from wearing a wearable data system. The circadian trajectory might be derived by a scheduler using at least one biophysics model of a human circadian clock and at least one a statistical model of the human circadian clock. The preferred treatment time period might be optimized based on associating a circadian time with a time for taking a drug for generating a raw model output.

A model output might be used by an environmental controller to adjust an environment such that circadian-relevant behaviors are adjusted towards a target-constrained time. Such target-constrained times might correspond to treatments administered at an infusion clinic or a scheduled surgery.

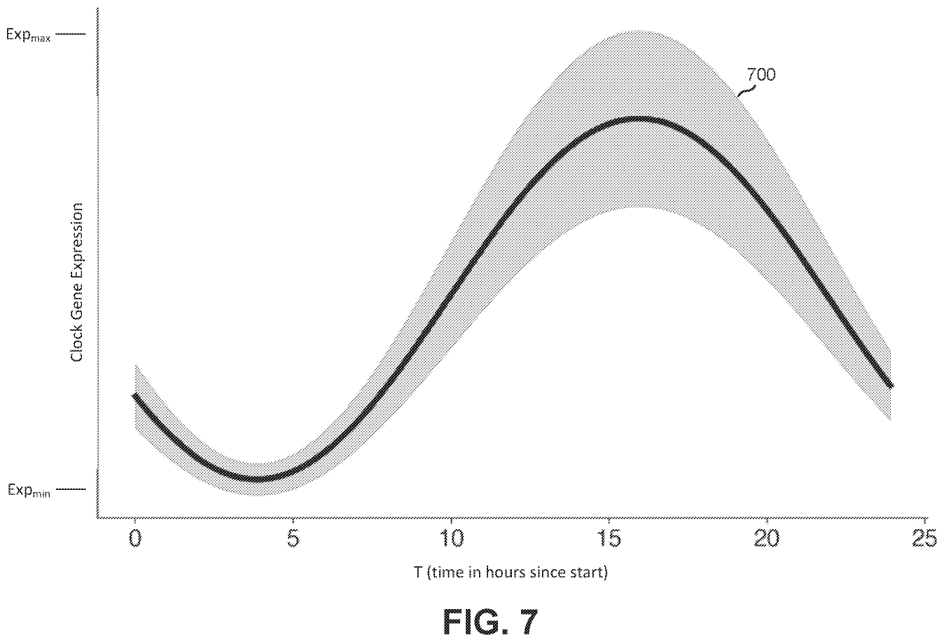

FIG. 7 illustrates an example of a trajectory 700 having some uncertainty built in. Uncertainty for each state in a trajectory can be calculated with a trajectory bundle. In FIG. 7, the uncertainty is illustrated by the lightly shaded uncertainty region 702.

The digital twin simulation can be carried out for varying inputs, with different trajectories generated for each input. The distribution of the trajectories at a given time can be used to define the uncertainty for each circadian state. The different inputs given can be different wearable inputs (e.g., assuming the sensors on a wearable device were inaccurate or incomplete) or different model parameters (e.g., light sensitivity). The inputs can be chosen randomly or systematically from a distribution. This distribution can be the uniform distribution, or it can be a distribution based on historical data from a user or a population of users.

Uncertainty might be due to wide variances among trajectories. For example, where a patient treatment system might have trajectories for several different input values, but lacks the input value or certainty over the input value, the trajectory might be uncertain, as reflected in the plot shown in FIG. 7. Uncertainty might relate to, for each circadian state, central or peripheral, a reflection of how confident an estimation of that state is. For example, at time tₓ, a concentration of a particular molecule could be between 3 and 4 picograms/ml and that variance might contribute some level of uncertainty for time tₓ. On the other hand, if the concentration was only known to be between 3 and 300 picograms/ml at time tₓ, that would be a higher uncertainty. Circadian uncertainty might increase in light of jet lag and travel, as the effects of jet lag/travel on circadian patterns for a specific individual might not be well-known. Uncertainty can increase in the presence of missing data and can decrease in the presences of large volumes of input data.

Circadian-mapping profiles that map efficacy, toxicity, etc. variances to circadian state can be developed by collecting examples from a large number of patients and processing that data into circadian-mapping profiles. These circadian-mapping profiles can be stored as data structures that are processable by the patient treatment system and might be used with trajectory data to make treatment timing decisions and/or recommendations.

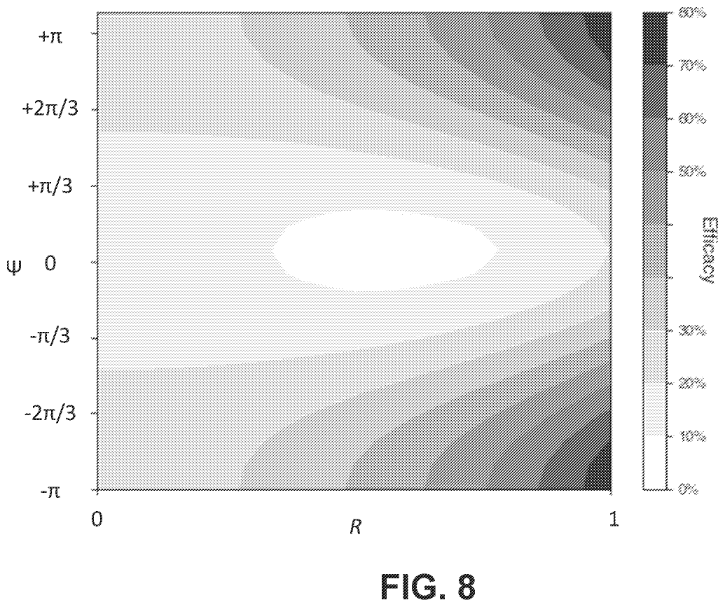

FIG. 8 illustrates an example circadian-mapping profile, an efficacy profile, that maps between circadian states to efficacy of a drug. The circadian-mapping profile can be represented and stored in memory using various data structures that a processor can use to determine, for a given circadian state, what an efficacy value might be or conversely, for a given efficacy value or range of values, which circadian state or range of states correspond to the given efficacy value or range of values. In the example of FIG. 8, R is a value that corresponds to how synchronized neurons in the SCN are and can range in this example from R=0 to R=1, while Ψ is a value that corresponds to a circadian state that might range from −π to +π (and represent a state at a point in time within a circadian cycle or trajectory.

Efficacy is shown in 10% ranges from 0% to 80%, with different shades of gray corresponding to particular ranges. Other ranges are possible. In this example of FIG. 8, at a point 802, corresponding to a circadian state of Ψ=+π/3 and an SCN neuron synchronization value of R=0.5, the efficacy would be between 10% and 20%. At another point 804, at a circadian state of Ψ=−2π/3 and an SCN neuron synchronization value of R=0.84, the efficacy would be between 40% and 50%. While FIG. 8 is a representation of what might be a circadian-mapping profile stored in computer memory, the computer representation might have more resolution and the value represented in the circadian-mapping profile for point 802 might be 14%. All other things being equal, this might indicate that the treatment might benefit from being adjusted closer to Ψ=+π and R=0.98.

While FIG. 8 illustrates a two-dimensional (2D) profile, a circadian-mapping profile might have more than one dimension, with one or more of the dimensions being components of the circadian state, or components of central and peripheral circadian states. For example, an efficacy profile might have one dimension for circadian state that is a central circadian state of an expression level of the Bmall gene, another dimension for drug efficacy for a given drug, a peripheral circadian state of an expression level of NHE3, a biological state of concentrations of drugs already in the system, etc. An N-dimensional circadian-mapping profile stored in memory as a data structure might provide a processor with indications of what an efficacy of the drug would be at a point or region of an N-dimensional space corresponding to a current expression level of the Bmall gene, a current expression level of NHE3, current concentrations of drugs already in the system, etc.

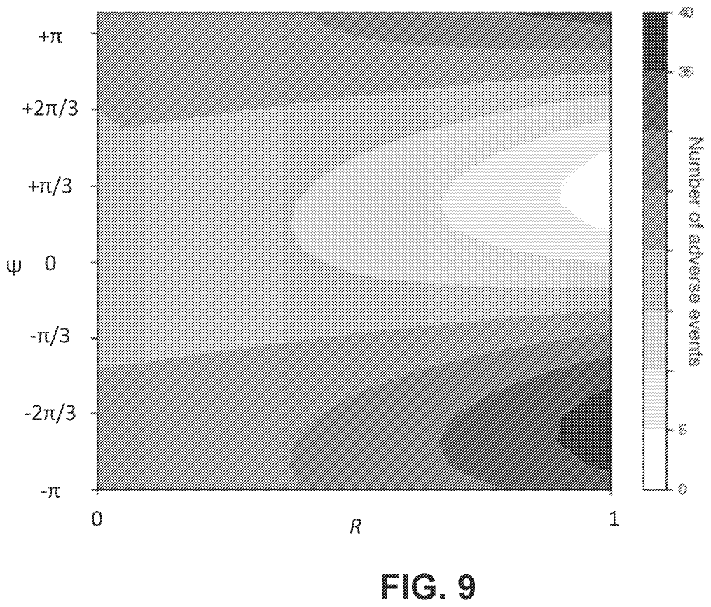

FIG. 9 illustrates another example circadian-mapping profile, this one a toxicity profile, that maps circadian states to a toxicity measure of a drug. The toxicity profile can be represented and stored in memory using various data structures that a processor can use to determine, for a given circadian state, what a toxicity value might be or conversely, for a given toxicity value or range of values, which circadian state or range of states correspond to the given toxicity value or range of values. This might be used by a patient management system to prompt a patient to initiate a treatment, such as the taking of a prescribed medication. As illustrated in FIGS. 8 and 9, some circadian states might be preferred over others for the initiation of a treatment and the methods described herein for inferring a current or future circadian state can be useful.

In the example of FIG. 9, R is a value that corresponds to how synchronized neurons in the SCN and Ψ is a value that corresponds to a circadian state, as with FIG. 8. For various values of those two axes, a range of adverse events is indicated, which can represent an indication of toxicity. The circadian-mapping profile can be represented and stored in memory using various data structures that a processor can use to determine, for a given circadian state, what an adverse event count value might be or conversely, for a given adverse event count value or range of values, which circadian state or range of states correspond to the given adverse event count value or range of values. In the example of FIG. 9, R can range from R=0 to R=1, while circadian state, Ψ, can range from −π to +π and represent a state at a point in time within a circadian cycle or trajectory.

The number of adverse events is shown in ranges of five events from 0 events to 40 events, with different shades of gray corresponding to particular ranges. Other ranges are possible. In this example of FIG. 9, at a point 902, corresponding to a circadian state of Ψ=+π/3 and an SCN neuron synchronization value of R=0.5, the number of adverse events would be indicated in the circadian-mapping profile as being between 10 to 15. At another point 904, at a circadian state of Ψ=−2π/3 and an SCN neuron synchronization value of R=0.84, the number of adverse events would be indicated in the circadian-mapping profile as being between 30 to 35. All other things being equal, this might indicate that the treatment might benefit from being adjusted closer to Ψ=+π/3 and R=0.95. While FIG. 9 is a representation of what might be a circadian-mapping profile stored in computer memory, the computer representation might have more resolution and the value represented in the circadian-mapping profile for point 904 might be 32.

In some implementations, selection of to Ψ and R might be optimized taking into account multiple circadian-mapping profiles, such as one or more efficacy profile and one or more toxicity profile to find a best fit when an optimal region of each circadian-mapping profile is not all at the same values of Ψ and R.

While FIG. 9 illustrates a two-dimensional (2D) profile, a circadian-mapping profile might have more than one dimension, with one or more of the dimensions being circadian components of the circadian state, or components of central and peripheral circadian states. For example, a toxicity profile might have one dimension for circadian state that is a central circadian state of an expression level of the Bmall gene, another dimension for drug toxicity for a given drug, a peripheral circadian state of an expression level of NHE3, a biological state of concentrations of drugs already in the system, etc. An N-dimensional profile stored in memory as a data structure might provide a processor with indications of what a toxicity of the drug would be at a point or region of an N-dimensional space corresponding to a current expression level of the Bmall gene, a current expression level of NHE3, current concentrations of drugs already in the system, etc.

As explained herein elsewhere, the patient treatment system might convert large numbers of circadian trajectories and clinical outcome data into efficacy and/or toxicity profiles. Such processing to generate circadian-mapping profiles might also take as inputs demographics data and other trajectories and output a circadian-mapping profile that maps circadian states (and possibly other inputs) to efficacy values, toxicity values, or other profile type values.

A treatment mapping can be represented in a data structure that provides a rule for converting a circadian trajectory with affiliated uncertainty and a circadian-mapping profile into a set of administration windows. An administration window can be narrower (e.g., “take a drug between 5:00 PM and 5:30 PM”) when the uncertainty is low. An administration window can be wider (e.g., “take a drug in the afternoon”) when the uncertainty is high. The treatment mapping can be computed from a patient's circadian trajectory, which in turn can be derived from wearable data. For instance, wearable data can be used to arrive at both a circadian trajectory and uncertainties for each point by simulating a digital twin of the suprachiasmatic nucleus (SCN). In this example, a circadian state can be represented by the firing rate and firing cohesion of the neurons in the SCN, or by the expression of genes in SCN cells in a moment of time.

A fixed treatment window might exist for a period of time when a treatment is scheduled that is out of the user's control. This can be a surgery or an infusion. Fixed treatment windows might be dealt with by instituting controls that can result in altering the mapping of circadian state to wall-clock time. A control might be a behavior or activity that affects biological states. A control might be represented in memory as a data structure that comprises an action and a time period. A control might be a pulse of light at a specific time to alter a light exposure history or a suggestion to do so (e.g., “Get 10,000 lux of light exposure for 45 minutes starting at 2:45 PM on October 3rd”), a signal to suggest exercising during a window of time (e.g., “Do a moderate work out from 11:45 AM to 12:45 PM”), a signal to take a drug during a window of time, etc.

A control system might consider a current circadian state, with affiliated uncertainty, as well as a fixed treatment window, and prescribe a set of controls to move a recommended administration window, which corresponds to a future circadian trajectory and an efficacy and/or toxicity profile, to overlap as much as possible with the fixed treatment window.

Accurately determining an individual patient's circadian time so that it can be used in the treatment mapping might include wearable data augmented by a data structure such as a work calendar data table. Inclusion of this data structure can reduce uncertainty.

The digital twin simulation for converting inputs into trajectories can address problems with masking caused by external factors that obscure true circadian signals to allow for accurate, personalized treatment timing recommendations. For instance, the digital twin simulation can address the problems caused by noise and outliers in a signal, which confound the estimation of true circadian time, by imposing realistic bounds on how much of an effect any time period of signal can have on the output circadian state. Without steps to address masking, administration window times can become highly volatile and inaccurate.

The administration windows may be presented as only timing recommendations for doses with a fixed amount, such as pills (e.g., “there is a good time of day to take this pill and for you specifically, that time of day is between around 10 AM to 11 AM local time tomorrow”), or they can be presented as both dosing and timing recommendations (e.g., “your morning dose should be X mg between around 10 AM to 11 AM local time and X+Y mg between around 12 PM and 1 PM”).

Inputs to a digital twin simulation used to arrive at a trajectory might be wearable device data streams, demographic data stored in a user data record, or demographic details (e.g., gender, age, geographic residence location, etc.).

Inputs to a circadian-mapping profile can include known optimal timings for the individual based on self-report or other records, a set of timing restrictions represented in a restrictions table or stored restrictions rule set (e.g., restrictions on time intervals between doses), and the like.

A user interface might be provided to inform the patient of administration windows and alerts at wall-clock times that correspond to administration windows.

A digital twin simulation may also incorporate environmental factor input and outputs and use those to modify a trajectory. This in turn can change the inputs to the treatment mapping, resulting in new administration windows. For instance, a hospital room could include known lighting levels in a digital twin simulation, reducing uncertainty for a trajectory and narrowing the administration windows presented to the user.

Environmental controls can also integrate with a control system. For example, a control system may interact with environmental controls to change an environment based on a set of rules or data that move future administration windows to overlap as much as possible with fixed treatment times. As an example, the environmental control system might alter light levels, light colors, heating, cooling, etc. in ways that would alter a patient's circadian trajectory so that the resulting treatment mapping recommends an administration window near or overlapping a fixed treatment time.

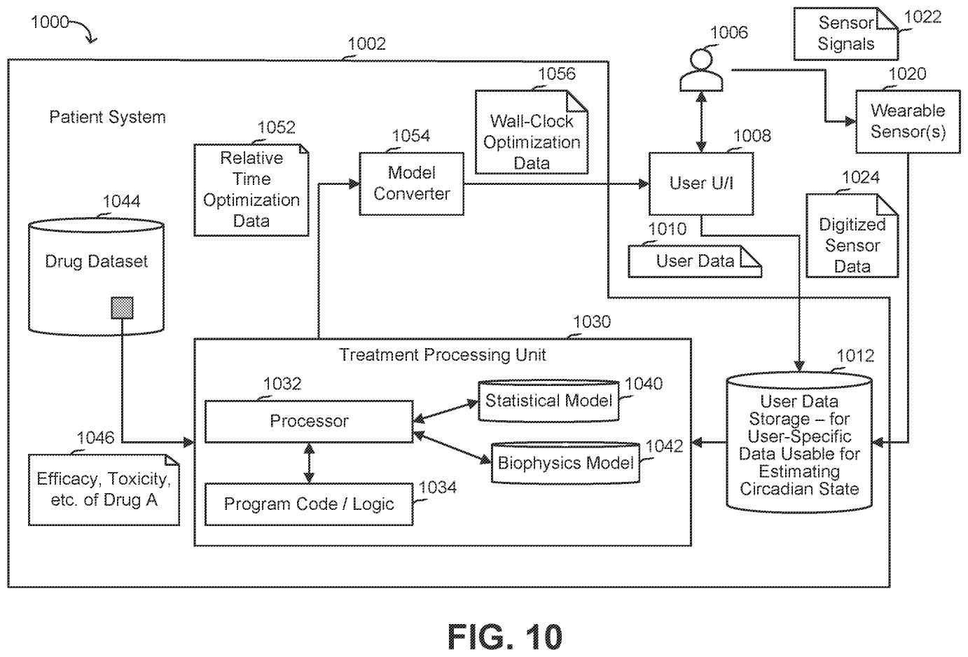

FIGS. 10-12 illustrate a hardware structure and examples that could be used to implement the patient treatment system described herein.

A treatment system can gather inputs, apply a treatment mapping, and provide a user interface to indicate, possibly at or near certain wall-clock times, computed administration windows. The administration windows may initially cover long windows of time (e.g., “all afternoon”) and then become more specific as uncertainty is reduced (“between 8 and 8:30 PM”).

As described herein, a treatment system can take in patient inputs, and other inputs, process a treatment mapping that intakes biological states and at least one circadian state, along with a circadian-mapping profile, producing administration windows which correspond to wall clock times. The display of the administration windows can be triggered near the time of the start of the administration window, and this display can be provided through a user interface that would issue a treatment administration message, such as a reminder to a patient or caregiver to administer the corresponding treatment.

FIG. 10 is a diagram of a patient treatment system 1000, according to various embodiments. Patient treatment system 1000 can be used for producing optimal administration windows related to a biological process and provide patient information related to treatments and timing of treatments. In one example of the treatment mapping to determine best timing, a best time for taking a drug can be seven hours after the circadian state corresponding to melatonin onset. In another example, the treatment mapping can include a complicated molecular model that has equations capturing how different molecules in the drug bind to the body and interact with each other in order to select the best administration window given these drug interactions. In another example, the treatment mapping can take demographics information such as ethnicity, sex, age, and other drugs as parameters for input. Logical gating may be used and in one example, the treatment mapping ensures drugs are spaced at least twenty-four hours apart. In a further example, where molecules bind and interact in the equations, the variables represent molecular equations and parameters represent binding rates. In many cases, indicating to a patient an optimal timing to take a drug or perform other treatments might depend on a determination of a circadian state and possibly also an uncertainty about the patient's circadian state. As with the example above, if the optimal time for taking a drug is seven hours after the patient has experienced melatonin onset, a patient system 1002 of patient treatment system 1000 might send a message around the time determined to be seven hours after melatonin onset. If there is uncertainty about when that is, the patient might be messaged around the start of a window of uncertainty and provided an indication of the uncertainty (e.g., “The best time to take drug A, for you, is 7 hours after melatonin onset. Based on an analysis of data measured from you and data input by you, melatonin onset today for you would have been at 11:25 AM with an uncertainty of around +/−35 minutes. You should therefore take Drug A, in the indicated dose, at 6:25 PM and given the uncertainty, the actual optimal time to take Drug A might be between 5:50 PM and 7:00 PM.”).

As illustrated in FIG. 10, a user/patient 1006 might provide a user interface 1008 with user data 1010 that is stored in user data storage 1012. User data 1010 might include data received from user 1006 via user interface 1008, such as user demographics, user work information, user travel information, indications of other medications or treatments, user sentiment (how the user is feeling, how the user has reacted to past treatments, etc.) and the like, as well as self-reported manually entered data, such as historical mealtimes, light exposure, exercise times, or lists of drugs the patient is currently taking. Other user-related data might be obtained by wearable sensors 1020 converting sensor signals 1022 into digitized sensor data 1024 that is provided to user data storage 1012. That user data can be provided to a treatment processing unit 1030. Digitized sensor data 1024 might include steps taken, heart rate, temperature, etc.

Treatment processing unit 1030 might comprise a processor 1032 and program code/logic 1034 that can be used to take in user-specific data to determine a current circadian state of the user, as well as state interaction information from molecular stimulations and data about efficacy and/or toxicity profiles of various drugs to determine an optimal or preferred circadian state for administering a treatment. Data provided to treatment processing unit 1030 can used to modify a statistical 1040 and/or a biophysics model 1042 to track circadian state. User details, such as a list of drugs the patient has reported as taking, can be used to determine which circadian-mapping profile to use from among a set of profiles.

The circadian state of the user can be passed on as a circadian-mapping profile for that patient and then an administration window can be passed to the user as an output. For example, as illustrated in FIG. 10, treatment processing unit 1030 can receive, from a drug data set 1044, data 1046 related to efficacy, toxicity, other drug facts, etc. of a particular treatment such as the administration of “Drug A”. Considering also the user-specific data about user/patient 1006, treatment processing unit 1030 can output a record 1052 representing a relative time optimization that would indicate a best time or time range for treatment for user/patient 1006 expressed in circadian relative time. For example, the best time might be represented as a number of hours before or after a defined circadian state, such as the circadian state corresponding to melatonin onset. A model converter 1054 could convert data in record 1052 into a wall-clock optimization data record 1056, which in turn can be provided to user interface 1008 to present a message or a signal to user/patient 1006 indicating the best, preferred, and/or optimal wall-clock time for administering a treatment such as taking a medication.

As explained in detail herein, circadian cycles might pass through various circadian states and the pattern of circadian states can be referred to as a circadian trajectory. A circadian trajectory might correspond to a particular physical state of a person's body (or perhaps over a population of people or other organisms that exhibit circadian cycles). While commonly represented as a simple sinewave having a frequency of around one day or slightly longer and thus circadian states that can be thought of as corresponding to phases of the simple sinewave, circadian states and circadian trajectories are often not so simply modeled. To account for this, apparatus and methods described herein can be used to predict or estimate a circadian trajectory of an organism and a mapping of circadian states along that circadian trajectory onto wall-clock time. This can be useful for timing treatments in which optimal circadian states along circadian trajectory for administering the treatments are known when a current circadian state of a user/patient and/or a future wall-clock time corresponding to a future circadian state of the user/patient needs to be determined.

Circadian trajectories might be reflected in concentrations of molecules that rise and fall with a circadian pattern. These trajectories might be called “peripheral circadian trajectories” when they occur in peripheral clocks, such as the biological clock mechanisms related to the stomach or the liver. These trajectories might be called “central circadian trajectories” when they occur in the central clock, the SCN. These trajectories, or estimates thereof, can be generated using digital twin simulations as they are often not easily determined from direct measurement of an organism. In one instance, digital twin simulations of circadian trajectories are generated by a processor according to a differential equation describing the concentration of the molecule, enabling the concentration to rise and fall in a dynamical, physiological manner.

Concentrations of molecules external to the body can be described by trajectories. These trajectories can be generated by digital twin simulations. In one instance, digital twin simulations of external molecule trajectories are generated by a processor according to a differential equation describing the concentration of the molecule, enabling the concentration to rise and fall in a dynamical, physiological manner. For instance, an impulse of caffeine to the system can be captured by a digital twin simulation in which the caffeine slowly drains from the bloodstream at a rate expressed as a differential equation.

The information provided to the treatment mapping can include data on pill consumption, such as a pill that automatically tracks that it has been collected, or detection of wrist motion via an accelerometer that is predicted to match the wrist motion of taking a pill. In this example, the treatment mapping can use the pill timing information to arrive at more accurate trajectories for the pill concentration in the body. It can also use this information to apply logical gating rules, such as spacing between the intake of two consecutive doses.

In yet another example, a treatment assignment and coordination system can be used to assist in scheduling for a clinic or hospital. In a treatment assignment and coordination system, a treatment mapping is repeatedly applied across a number of individuals to assist a clinic or hospital schedule people for appointments (infusions, surgeries) to arrive at recommendations for fixed treatment windows (e.g., clinical or surgery time slots for appointments) that best align with the individuals' administration windows. For instance, the treatment mapping can be applied to determine the administration windows for all people at a clinic, and the people with the earlier administration windows can be assigned to earlier fixed treatment windows. The collective raw treatment mapping output is converted into a human interpretable form being a display of best time for surgery or clinic visit amongst available time slots.

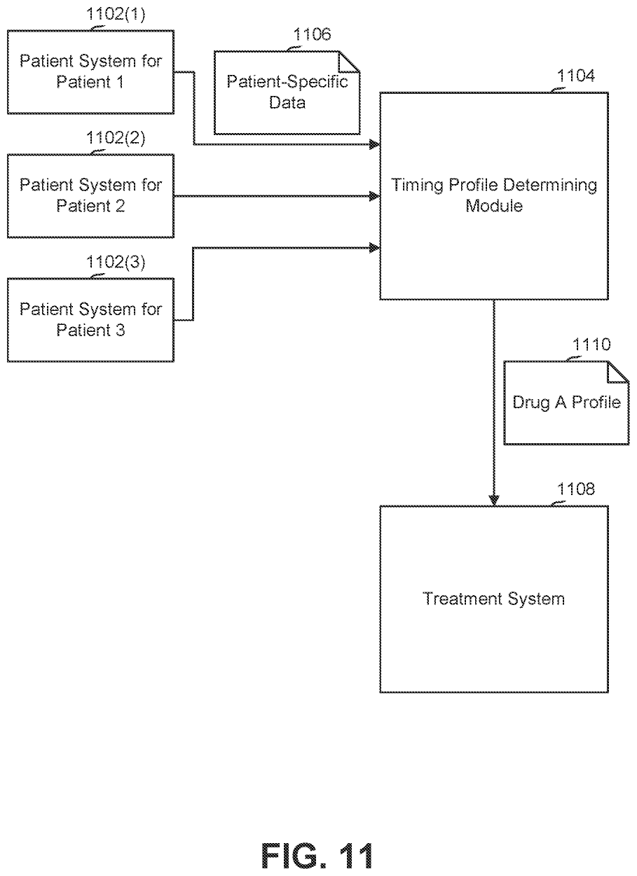

FIG. 11 is a diagram of a circadian-mapping profile identification system. As shown there, circadian-mapping profiles, such as efficacy and/or toxicity profiles, are generated from circadian trajectories and outcomes data from multiple individuals, who either self-report such data or have such data automatically detected. As shown in FIG. 11, there might be several patient systems 1102 for multiple patients. Each patient system 1102(i) might provide, to a timing profile determining module 1104, circadian trajectories and outcomes data that is maintained for patient i, such as patient-specific data 1106 from each of patient systems 1102. Timing profile determining module 1104 can then supply a drug profile data record 1110 to a treatment system 1108. An example of patient-specific data might be “Adverse events for Patient ABC12 include incident IN0045371 in which the patient dosed at 7:34 PM, their circadian state at the time of dosing was 0.243813 (along a trajectory that is scaled from 0.0 to 1.0), and the patient self-reported two headaches the next day.” If, for example, a large number of patients reported next-day headaches and a large number did not and a significant distinguishing factor between the two groups is that those who dosed having a circadian state of between 0.2 and 0.3 at the time of dosing are in the first group (the group having the adverse effect) and those who dosed having a circadian state of between 0.6 and 0.7 at the time of dosing are in the second group (the group reporting no adverse effect), then it might be expected that drug profile data record 1110 would indicate for Drug A that a circadian state of 0.6 to 0.7 is preferred for administering Drug A.

Patient-specific data 1106 passed into timing profile determining module 1104 could be circadian state data over time, as well as efficacy and/or toxicity outcomes. These could include efficacy outcomes, like remission rate, tumor shrinkage, overall survival or changes in blood pressure, lipid profiles, or other biological outcomes. These could also be toxicity outcomes, such as number of adverse events or side-effects reported. Timing profile determining module 1104 can aggregate the data from multiple patients to arrive at a tool for converting circadian states and other inputs into efficacy and toxicity.

The cumulative data generates an efficacy and/or toxicity profile that takes circadian states, as well as other possible inputs, and yields efficacy and/or toxicity. For instance, a circadian-mapping profile identification system could identify that the reason an 8 AM dosing reduces blood pressure for one person while having no effect for a different person is because the two individuals are at different circadian states at 8 AM of wall-clock time. The system could then learn a relationship between circadian states, taken from computed trajectories, and efficacy, and in doing so reveal trends that are not obvious from the wall-clock time of administration. For example, the system could produce an efficacy profile that converts a circadian state (such as peak Bmall expression) into an efficacy fraction (80% of peak efficacy at peak Bmall expression).

This circadian-mapping profile identification system could identify gender differences and include them as optional inputs in the circadian-mapping profile. For instance, peak Bmall expression could correspond to 80% efficacy for men and 70% efficacy for women. It could also identify age-related effects and include them as optional inputs in the resulting circadian-mapping profile. It could also identify demographics effects and include them as optional inputs in the resulting circadian-mapping profiles.

This circadian-mapping profile identification system could identify the effects of meal interactions and include them as optional inputs to the circadian-mapping profile. The circadian-mapping profile identification system can build circadian-mapping profiles from self-report data or from signals directly extracted from the user. For instance, the system could identify circadian states affiliated with lower blood pressure without user manual input. The circadian-mapping profile identification system could also be used to extract the best circadian timing for a treatment to reduce self-reported side-effects of the treatment.

In addition to, or instead of, administering a treatment, the treatment system can include a control system, a trigger from which might be an indicator of a control. A control can be an action the patient is to take, such as performing an exercise routine at a specific time, to assist the individual to move their administration window to overlap with a fixed treatment window. A control can also be used to reduce uncertainty, which can narrow the width of an administration window.

An efficacy profile can be generalized to include non-drug and non-medical treatment activities. For instance, an efficacy profile could relate exercise at different circadian states to efficacy at reducing diabetes risk.

An administration window can be generalized to include non-drug and non-medical treatment activities. For example, an administration window could recommend exercise, meal timing and composition at a specific time to target lowering diabetes risk, according to an efficacy profile. A different efficacy profile could yield administration windows for exercise at different times if one's goals relate to improving hypertension, for instance.

An administration window can also be generalized to include vaccine timings and other shots.

An administration window could also be applied for recommended timing for fertility treatments such as in vitro fertilization (IVF).

An administration window can also be generalized to include times to eat or to avoid eating, as well as the composition and/or size of the meal. For example, for the control of blood sugar the administration window could give a meal timing window as well as the protein, fats and carbohydrate composition for the meal.

An administration window can be large, such as six to twelve hours in duration. In such a case, the specific timing of the administration window might be of most significance to shift workers, recent travelers, and others who have recently experienced significant shifts in their circadian trajectories, for whom concepts like biological morning and night are poorly defined.

This system's applications are not limited to timing pill consumption. The system can used to identify the best fixed treatment windows for a user, e.g., for an infusion, or the best time for surgery. The system can instruct a person the best time to go into a clinic out of all the times the clinic is available, or from amongst the available appointment times at the clinic.

The system can inform people the best time for scheduling a surgery. On the other hand, the system can inform people on what controls are best for them in the lead up to surgery in order to allow for better recovery times from surgery. These controls can be actions the user should take. For example, if a toxicity profile indicates that the patient will recover more easily if surgery occurs during circadian states that habitually happen for this patient during the night, but the surgery is scheduled for the day, that person's circadian trajectory can be shifted by the controls recommended by the control system to make the circadian states that habitually happen during the night shift earlier or later, to better overlap with the fixed treatment window during daytime hours.

In another example, the treatment system can interface with digital therapeutics in pharmaceuticals. The treatment system may be integrated into a feedback loop system in which sensors embedded in the drug or in the pill bottle can track drug consumption. The sensors can provide information about what wall-clock time patients are taking the drug. This information can be provided to the treatment mapping to inform logical gating rules for spacing drug consumption, changing the recommended administration windows. This information can also be used to inform trajectories calculated using a digital twin simulation which express how much of the drug is present in the body over time. These trajectories in turn can be used as inputs to the treatment mapping to carry out more sophisticated determination of administration windows.

In yet another example, the system serves as a tool for pharmaceutical companies to identify circadian-mapping profiles in their assets. Drug timing information can be retrieved directly from the drug or a patient's self-report timing in combination with a circadian-mapping profile identification system such as described herein, can generate circadian-mapping profiles for new assets, thereby enabling more new drugs to emerge from early-stage drug trials. These circadian-mapping profiles would map circadian states, and possibly other inputs such as biological states or demographic information, to efficacy and/or toxicity.

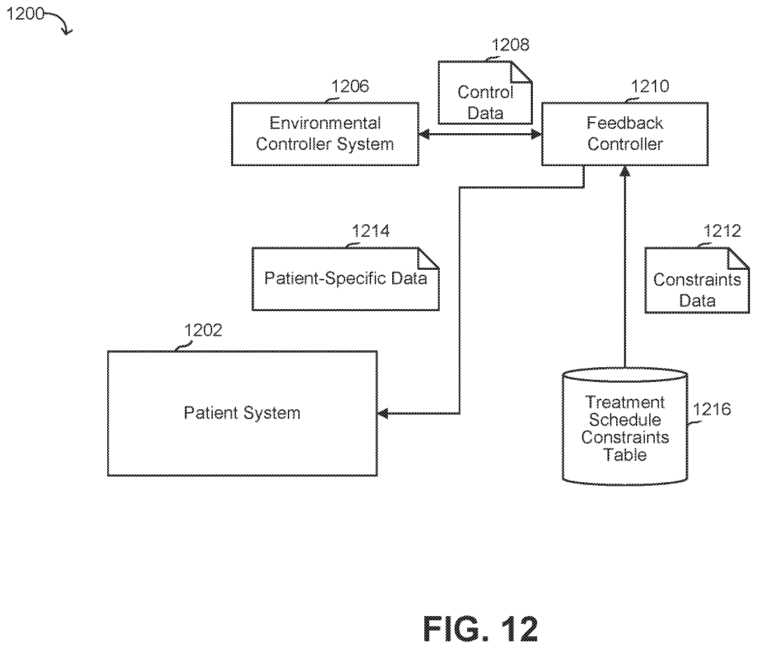

FIG. 12 is a diagram of a control system 1200, according to various embodiments. In an example, control system 1200 can be used to adjust someone's circadian trajectory through environmental controllers, such as light exposure via an LED device or behavioral nudges via a GUI, so their recommended administration windows overlap as much as possible with the fixed treatment windows. Control system 1200 might comprise a patient system 1202 that provides patient-specific data 1214 to a feedback controller 1210. Feedback controller 1210 might obtain constraints data 1212 from a treatment schedule constraints table 1216 to determine what timing constraints there might be on treatments. Patient-specific data 1214 might include the patient's current circadian trajectory and state, their current prescriptions, etc.

Treatment schedule constraints table 1216 could be used to provide a list of times for light exposure and other environmental controls for a given efficacy/toxicity profile and circadian state, as well as constraints data 1212, such as when a particular surgery has to happen. Patient system 1202 might provide a predicted circadian state. Feedback controller 1210 can then combine light exposure instructions, as well as the circadian state from patient system 1202, and trigger changes in the environmental control to move the patient's circadian state to better align with what is prescribed by the treatment schedule constraints schedule. Feedback controller 1210 might provide control data 1208 to an environmental controller system 1206 that can control an environment so as to influence a circadian trajectory.

Control system 1200 can identify information such as a phase response curve. In a phase response curve, “phase advance” and “phase delay” regions are identified during which light either pushes the clock forward or pushes the clock backwards. Control system 1200 can identify phase advance and phase delay regions for an individual and can provide light during the periods that better align the recommended administration windows with the fixed treatment time. Control data 1208 might be specific instructions, such as “turn on lights at 4:45 AM” in order to shift the patient into an earlier portion of their circadian trajectory.

The timing system can be integrated into a smart drug delivery system. For instance, it can be integrated into a time-lock pill system. The pill system could automatically release drugs each data according to the administration windows produced by the treatment mapping.

The timing system can be integrated into an artificial organ. The treatment system could identify administration windows for optimal compound release from the artificial organ system to best match the functioning of the organ the artificial organ is replacing. In this case, the efficacy profile is the circadian-mapping efficacy profile that best matches the original functioning of the organ (e.g., release at this circadian state has high efficacy because it resembles the original organ the best).

The timing system can be integrated into implantable pharmacies. The implantable pharmacy can release drugs according to administration windows set by the treatment system.

The control system can be integrated into implantable pharmacies. The implantable pharmacy can release drugs to shift a circadian trajectory so the administration windows shift to more overlap a fixed treatment time.

### Example Components

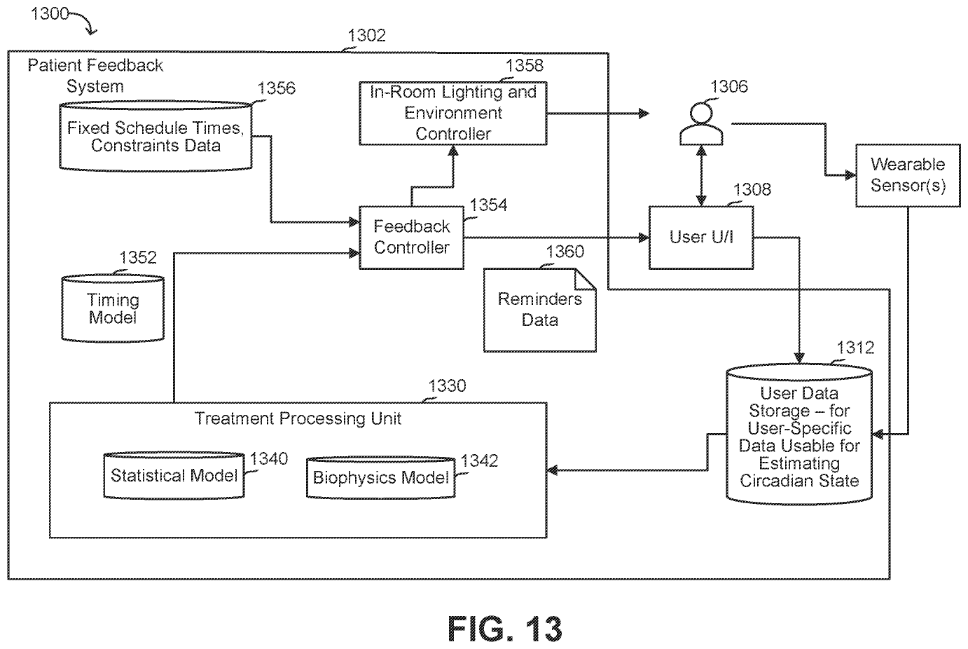

FIG. 13 illustrates components that might be used in a control system 1300 to change environmental cues and behavioral triggers in response a fixed treatment time and user data, according to various embodiments. FIG. 13 is a simplified figure and boxes therein might represent data structures, computational elements, sensors, and/or users. In some embodiments, elements shown that are similar to elements of FIG. 10 might operate similarly. As illustrated in FIG. 13, control system 1300 might include a patient feedback system 1302 for providing outputs to adjust a trajectory to meet some treatments schedule constraints.

A patient 1306 might have some treatment timing constraints, which might be represented a dataset 1356 that is coupled to a feedback controller 1354. Inputs from patient 1306, such as wearable sensor data and user-provided data, provided possibly via a user interface 1308, and stored in a user data storage 1312, can be provided to a treatment processing unit 1330. Sensor signal data might come from wearable sensor devices and/or environmental sensors of patient 1306. Environmental sensors might be used to detect a lighting environment, temperature environment, or other environment details the user is experiencing. Treatment processing unit 1330 might use digitized sensor data along with a circadian-mapping profile to design a series of environmental shifts that move the patient's circadian state so the administration window better aligns with the fixed schedule treatment time. Treatment processing unit 1330 that might derive the circadian state from a machine learning subsystem.

The machine learning subsystem might comprise a processor, storage 1340 for a statistical model and storage 1342 for a biophysics model that are both trained by the processor using training data corresponding to the sensor data. Statistical models can be machine learning models that are trained on wearable time series of data streams, such as light, activity, temperature, and heart rate, to predict gold standard biomarkers of circadian rhythms, such as dim light melatonin onset time, the concentration of melatonin over time, or core body temperature. The biophysics model can be human-generated or derived from data and can be created to match the physics of the suprachiasmatic nucleus; for instance, predicting firing rates in the ventral and dorsal SCN at any point in time, which can then be mapped to outputs like timing of dim light melatonin onset.

Treatment processing unit 1330 might output a circadian timing model 1352. The statistical model, the biophysics model, and circadian-mapping profile might be accessible to treatment processing unit 1330 in a computer memory possibly structured as neural network weights or another data structure that is usable as a trained machine learning model. Feedback controller 1354 can use a circadian-mapping profile as an input, as well as being provided with dataset 1356 indicating scheduled treatment times. One operation of patient feedback system 1302 is for feedback controller 1354 to output signals and/or data corresponding to environmental outputs, behavioral reminders, and the like that are tuned to adjust the user's circadian state to better align with the scheduled treatment times. The signals can be output to an environmental settings controller that might control in-room lighting by sending instructions to an in-room lighting and environment controller 1358. Feedback controller 1354 might send commands to some other environmental system that can control some aspect of the patient's environment.

For example, the control system could adjust in-room lighting to attempt to steer the user's circadian state towards alignment with the scheduled treatment times. The control system might also cause the display or issuance of behavioral reminders 1360 to the user. For example, the control system might cause a user's handheld device (e.g., via user interface 1308) to display a message such as “Your surgery is scheduled for 2:30 PM tomorrow. Since outcomes for that surgery are statistically improved for patients who undergo this surgery in mid-morning, you should remain active this evening later than usual and sleep in. I'll control the lighting to help with that.” or “You have an outpatient procedure at 9:30 AM tomorrow involving taking some medicine two hours before the procedure. Since better outcomes tend to occur when your body thinks it is an hour into your waking day, you should sleep now so you can rise around 6:30 AM.” In the latter example, the control system might have been altering lighting during the evening to reduce an amount of blue light to assist with aligning the wall clock evening with an evening portion of the user's circadian trajectory.

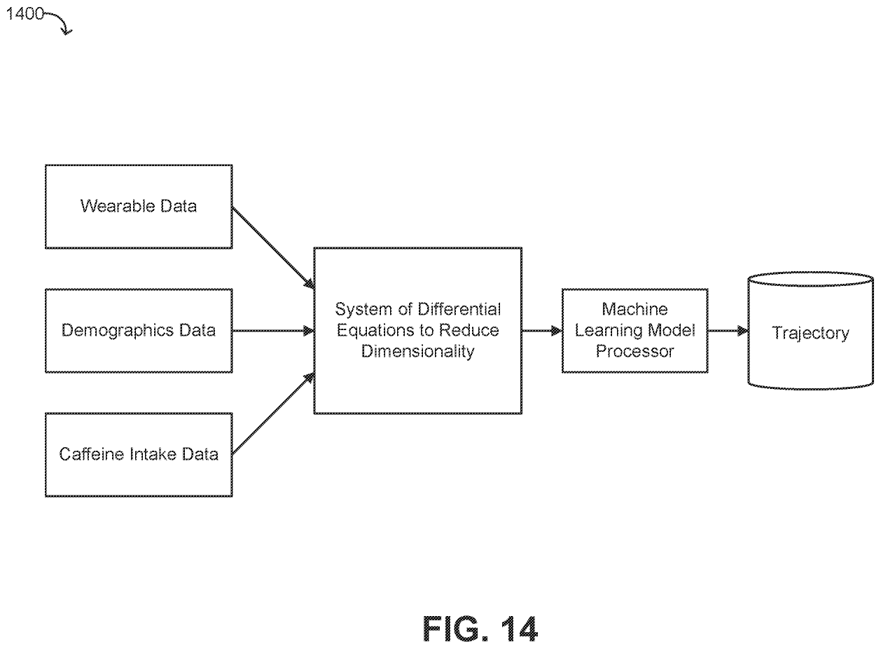

FIG. 14 illustrates components that might be used for a digital twin simulation, according to various embodiments. In an example digital twin simulation, a biophysics model representing a system of differential equations provides input into a machine learning model, which in turn yields a trajectory.

In the example shown, a simulator 1400 obtains data structures corresponding to sensor ratings, activities, and/or user data. For example, a simulator might read data from a user's wearable devices, read in demographics data from a user database, and read in patterns of caffeine intake (perhaps based on user manual input of caffeine intake, coordination with a purchasing app, coordination with a mapping app, or other information source) and then apply that data to a collection of differential equations to generate a dataset with reduced dimensionality. The simulator could then use that dataset and a trained machine learning model to generate a circadian trajectory for the digital twin and other portions of control system could use that generated circadian trajectory as a proxy for the user's circadian trajectory.

In the detailed example shown in FIG. 14, a distiller 1406 receives wearable data 1408, demographics data 1412, and caffeine intake records 1414 and is able to distill that into data can be provided to a model generator 1410. Model generator 1410 can then generate an ML model 1416, which can be provided to a trajectory generator 1420. Trajectory generator 1420 can then output and store a stored trajectory representation 1422.

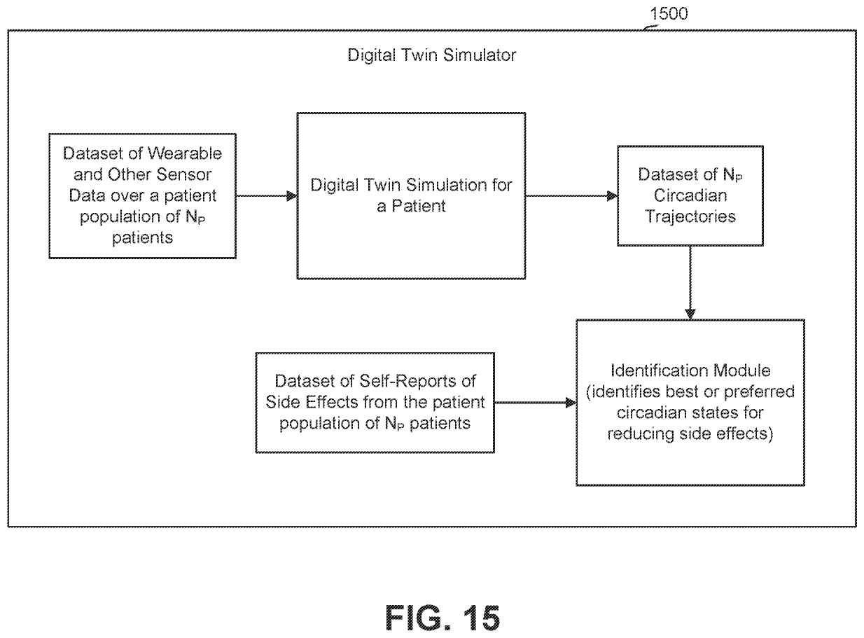

FIG. 15 illustrates components that might be used for a profile identification system 1500, according to various embodiments. As illustrated there, a collection of wearable data from a large population, such as 1000 people, 10,000 people, or more, might be provided to a digital twin simulator that would then generate a circadian trajectory for each member of the population. Profile identification system 1500 might also read data from a database of self-reported side effects reported by members of the population. The digital twin simulation might include a statistical model, a biophysics model, or both to process the wearable data to yield trajectories. Considering both the simulated trajectories and the reported side effects, the profile education system might be able to output data indicating which circadian states are good for reducing side effects and which circadian states are not. That output data might be represented as a toxicity profile.

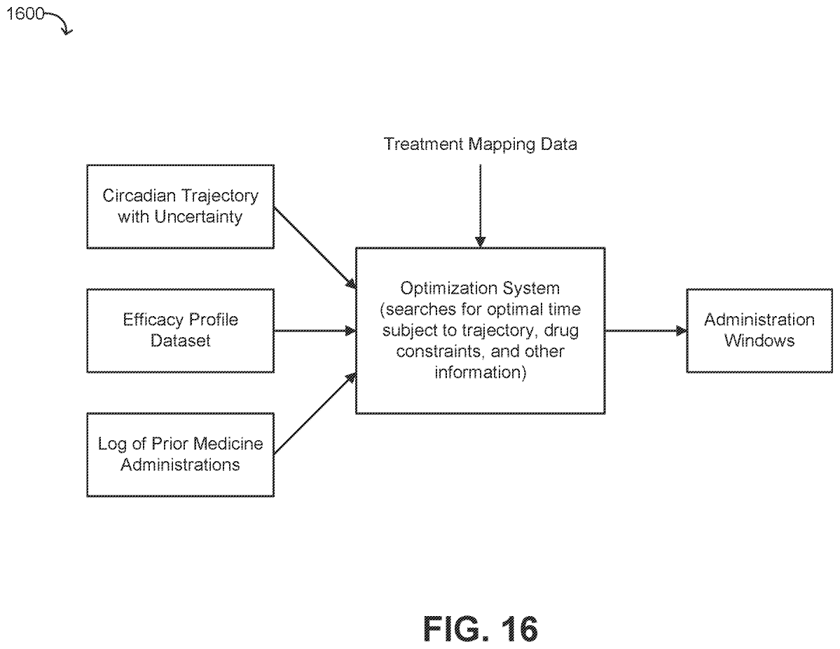

FIG. 16 illustrates components that might be used for a treatment mapping module 1600, according to various embodiments. In this example, treatment mapping module 1600 includes an optimization system that can read in a data structure representing a circadian trajectory, which may have some uncertainty such as that shown in FIG. 4, another data structure representing an efficacy profile dataset, and another dataset comprising a log of prior medicine administrations. Using that data, treatment mapping module 1600 might generate a dataset or transmit data that indicates preferred administration windows. Another input into the optimization system is treatment mapping data.

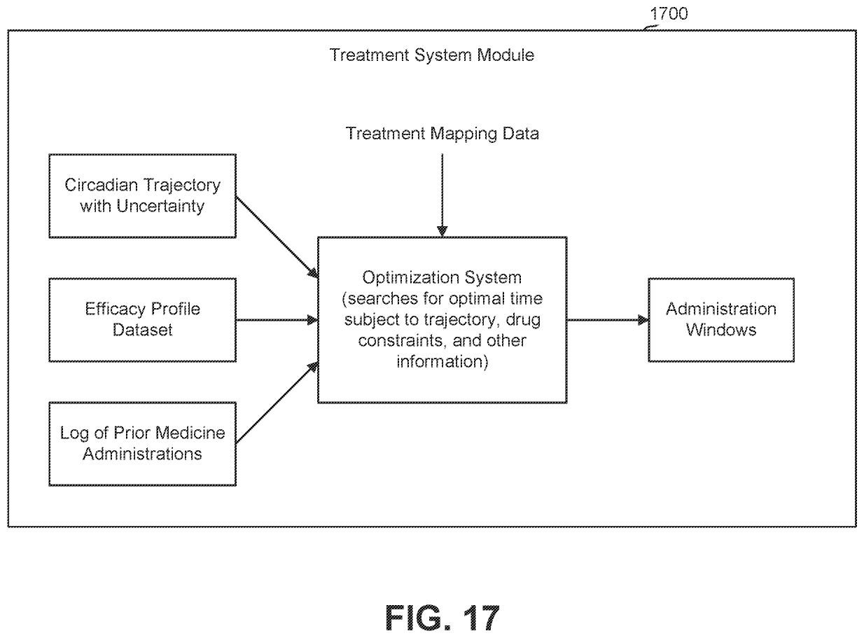

FIG. 17 illustrates components that might be used for a treatment system module 1700, according to various embodiments. As illustrated there, treatment system module 1700 might provide functionality similar to that of treatment mapping module 1600 in FIG. 16.

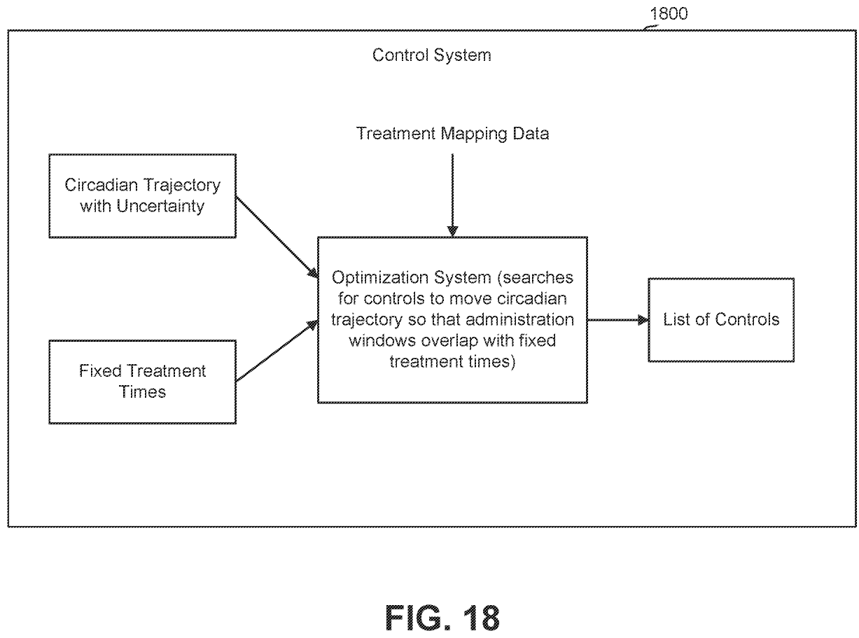

FIG. 18 illustrates components that might be used for a control system 1800, according to various embodiments. As illustrated there, control system 1800, as might be used as control system 1300 shown in FIG. 13, can take in a dataset representing a circadian state with uncertainty and a dataset representing treatment times and can output a dataset representing a list of controls, such as environmental light or behavioral changes, that can be output. Those outputs might be selected in order to change a user's circadian state to align their best administration window with a fixed treatment time.

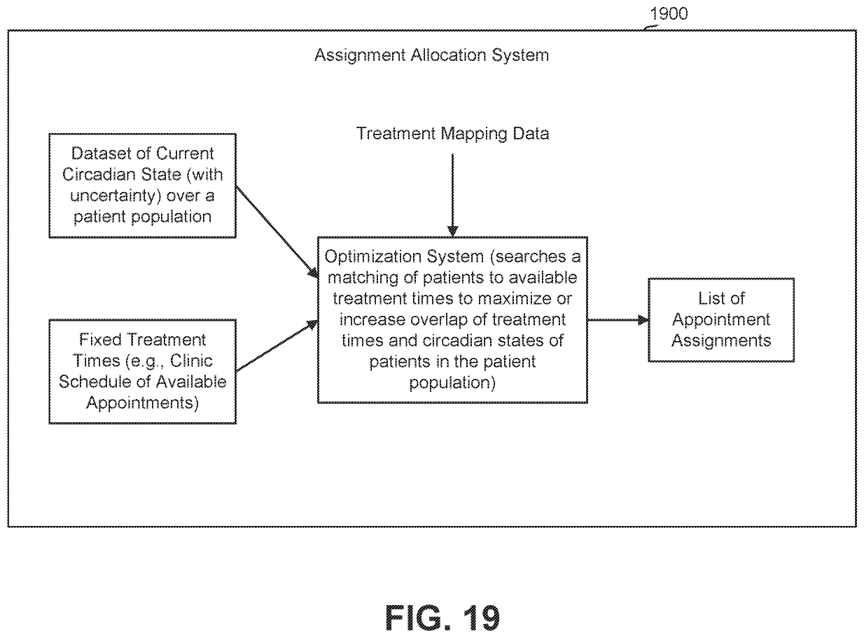

FIG. 19 illustrates components that might be used for a treatment assignment and coordination system 1900, according to various embodiments. In it, circadian state data for many people is mapped into a schedule which could assign each person to the fixed treatment time that most overlaps with their administration window, subject to the constraints of assigning each person to a treatment window and other desired parameters, such as the person's availability and timing preferences. Treatment assignment and coordination system 1900 could take in a dataset of circadian states or trajectories over a patient population (possibly with some uncertainty) of patients needing a particular treatment at a clinic having limited available appointments. Treatment assignment and coordination system 1900 could take in a dataset of available appointments. From that information, treatment assignment and coordination system 1900 could generate a list of appointment assignments. The appointments might be assigned to patients to maximize or increase overlap or alignment of treatment times and circadian states patients in the population based on what is known about preferred mappings of treatments to circadian states.

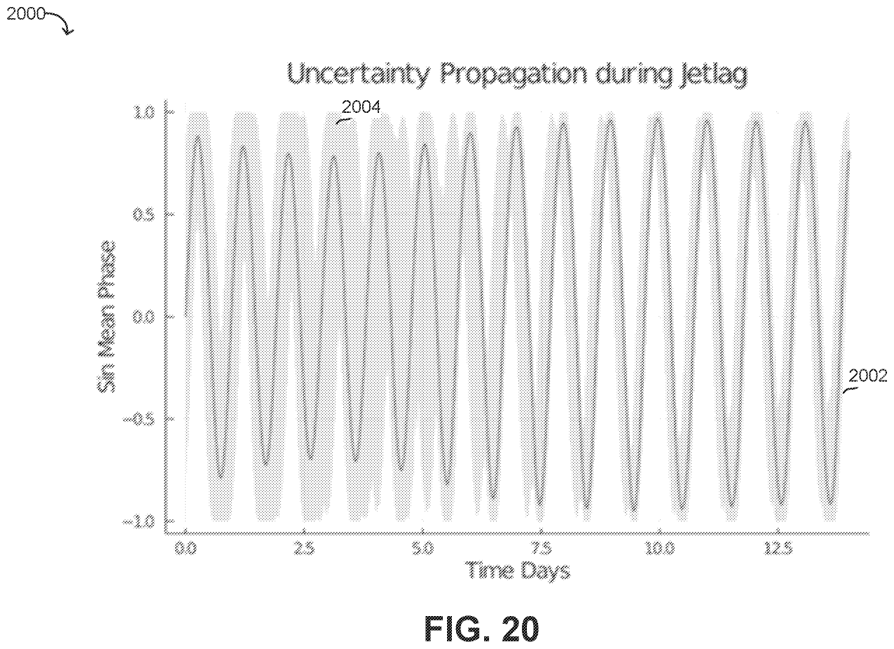

FIG. 20 illustrates another example of a trajectory uncertainty, according to various embodiments. In this specific example, a plot 2000 illustrates a baseline estimate 2002 (dark line) of a circadian state at any given time with uncertainty 2004 (light shading). In this example, the circadian trajectory has a strong sinusoidal component and a large amount of uncertainty over the first four days. In this example, traveling across several time zones is assumed prior to day 0 and might be the cause of the uncertainty. However, after a number of days, such as after 7.5 days, the uncertainty is greatly reduced. Various systems described herein can take into account how uncertainty can grow during periods of significant circadian disruption, while shrinking during periods of circadian stability. In this example, uncertainty of circadian state due to jet lag becomes more certain over time as the person remains in one time zone.

### Hardware Components

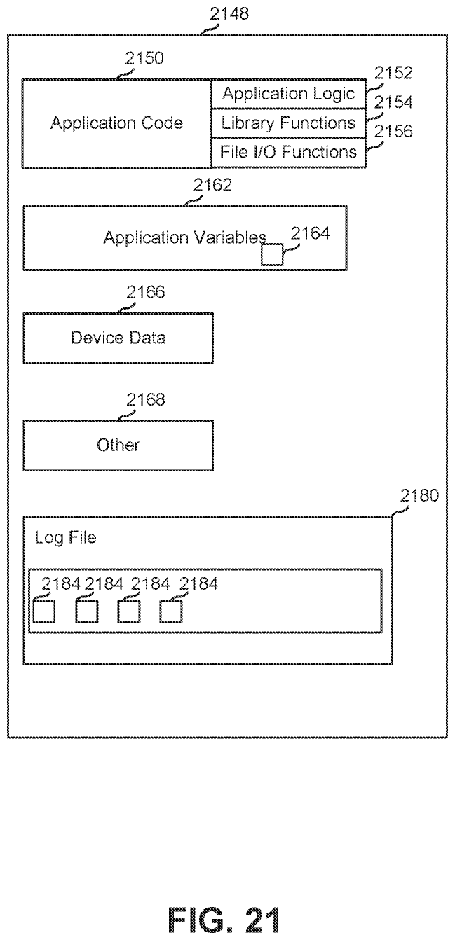

FIG. 21 is a simplified functional block diagram of a storage device 2148 having an application that can be accessed and executed by a processor in a computer system as might be part of embodiments of a patient treatment system and/or a computer system that performs the computations needed for treatment optimization. FIG. 21 also illustrates an example of memory elements that might be used by a processor to implement elements of the embodiments described herein. In some embodiments, the data structures are used by various components and tools, some of which are described in more detail herein. The data structures and program code used to operate on the data structures may be provided and/or carried by a transitory computer readable medium, e.g., a transmission medium such as in the form of a signal transmitted over a network. For example, where a functional block is referenced, it might be implemented as program code stored in memory. The application can be one or more of the applications described herein, running on servers, clients or other platforms or devices and might represent memory of one of the clients and/or servers illustrated elsewhere.

Storage device 2148 can be one or more memory device that can be accessed by a processor and storage device 2148 can have stored thereon application code 2150 that can be configured to store one or more processor readable instructions, in the form of write-only memory and/or writable memory. The application code 2150 can include application logic 2152, library functions 2154, and file I/O functions 2156 associated with the application. The memory elements of FIG. 21 might be used for a server or computer that interfaces with a user, generates data, and/or manages other aspects of a process described herein.

Storage device 2148 can also include application variables 2162 that can include one or more storage locations configured to receive input variables 2164. The application variables 2162 can include variables that are generated by the application or otherwise local to the application. The application variables 2162 can be generated, for example, from data retrieved from an external source, such as a user or an external device or application. The processor can execute the application code 2150 to generate the application variables 2162 provided to storage device 2148. Application variables 2162 might include operational details needed to perform the functions described herein.

Storage device 2148 can include storage for databases and other data described herein. One or more memory locations can be configured to store device data 2166. Device data 2166 can include data that is sourced by an external source, such as a user or an external device. Device data 2166 can include, for example, records being passed between servers prior to being transmitted or after being received. Other data 2168 might also be supplied.

Storage device 2148 can also include a log file 2180 having one or more storage locations 2184 configured to store results of the application or inputs provided to the application. For example, the log file 2180 can be configured to store a history of actions, alerts, error message and the like.

According to some embodiments, the techniques described herein are implemented by one or more generalized computing systems programmed to perform the techniques pursuant to program instructions in firmware, memory, other storage, or a combination. Special-purpose computing devices may be used, such as desktop computer systems, portable computer systems, handheld devices, networking devices or any other device that incorporates hard-wired and/or program logic to implement the techniques.

One embodiment might include a carrier medium carrying data that includes data having been processed by the methods described herein. The carrier medium can comprise any medium suitable for carrying the data, including a storage medium, e.g., solid-state memory, an optical disk or a magnetic disk, or a transient medium, e.g., a signal carrying the data such as a signal transmitted over a network, a digital signal, a radio frequency signal, an acoustic signal, an optical signal or an electrical signal.

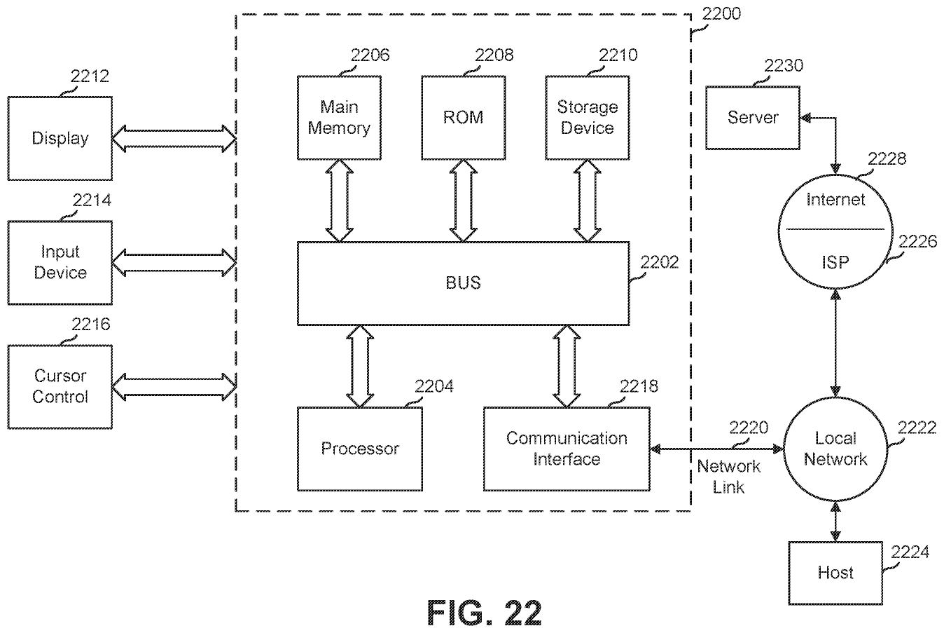

FIG. 22 is a block diagram that illustrates a computer system 2200 upon which the computer systems of the systems described herein and/or data structures shown in FIG. 21 may be implemented. Computer system 2200 includes a bus 2202 or other communication mechanism for communicating information, and a processor 2204 coupled with bus 2202 for processing information. Processor 2204 may be, for example, a general-purpose microprocessor.

Computer system 2200 also includes a main memory 2206, such as a random-access memory (RAM) or other dynamic storage device, coupled to bus 2202 for storing information and instructions to be executed by processor 2204. Main memory 2206 may also be used for storing temporary variables or other intermediate information during execution of instructions to be executed by processor 2204. Such instructions, when stored in non-transitory storage media accessible to processor 2204, render computer system 2200 into a special-purpose machine that is customized to perform the operations specified in the instructions.

Computer system 2200 further includes a read only memory (ROM) 2208 or other static storage device coupled to bus 2202 for storing static information and instructions for processor 2204. A storage device 2210, such as a magnetic disk or optical disk, is provided and coupled to bus 2202 for storing information and instructions.

Computer system 2200 may be coupled via bus 2202 to a display 2212, such as a computer monitor, for displaying information to a computer user. An input device 2214, including alphanumeric and other keys, is coupled to bus 2202 for communicating information and command selections to processor 2204. Another type of user input device is a cursor control 2216, such as a mouse, a trackball, or cursor direction keys for communicating direction information and command selections to processor 2204 and for controlling cursor movement on display 2212. This input device typically has two degrees of freedom in two axes, a first axis (e.g., x) and a second axis (e.g., y), that allows the device to specify positions in a plane.

Computer system 2200 may implement the techniques described herein using customized hard-wired logic, one or more ASICs or FPGAs, firmware and/or program logic which in combination with the computer system causes or programs computer system 2200 to be a special-purpose machine. According to one embodiment, the techniques herein are performed by computer system 2200 in response to processor 2204 executing one or more sequences of one or more instructions contained in main memory 2206. Such instructions may be read into main memory 2206 from another storage medium, such as storage device 2210. Execution of the sequences of instructions contained in main memory 2206 causes processor 2204 to perform the process steps described herein. In alternative embodiments, hard-wired circuitry may be used in place of or in combination with software instructions.

The term “storage media” as used herein refers to any non-transitory media that store data and/or instructions that cause a machine to operation in a specific fashion. Such storage media may include non-volatile media and/or volatile media. Non-volatile media includes, for example, optical or magnetic disks, such as storage device 2210. Volatile media includes dynamic memory, such as main memory 2206. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, an EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire, and fiber optics, including the wires that include bus 2202. Transmission media can also take the form of acoustic or light waves, such as those generated during radio-wave and infra-red data communications.

Various forms of media may be involved in carrying one or more sequences of one or more instructions to processor 2204 for execution. For example, the instructions may initially be carried on a magnetic disk or solid-state drive of a remote computer. The remote computer can load the instructions into its dynamic memory and send the instructions over a network connection. A modem or network interface local to computer system 2200 can receive the data. Bus 2202 carries the data to main memory 2206, from which processor 2204 retrieves and executes the instructions. The instructions received by main memory 2206 may optionally be stored on storage device 2210 either before or after execution by processor 2204.

Computer system 2200 also includes a communication interface 2218 coupled to bus 2202. Communication interface 2218 provides a two-way data communication coupling to a network link 2220 that is connected to a local network 2222. For example, communication interface 2218 may be a network card, a modem, a cable modem, or a satellite modem to provide a data communication connection to a corresponding type of telephone line or communications line. Wireless links may also be implemented. In any such implementation, communication interface 2218 sends and receives electrical, electromagnetic, or optical signals that carry digital data streams representing various types of information.

Network link 2220 typically provides data communication through one or more networks to other data devices. For example, network link 2220 may provide a connection through local network 2222 to a host computer 2224 or to data equipment operated by an Internet Service Provider (ISP) 2226. ISP 2226 in turn provides data communication services through the world-wide packet data communication network now commonly referred to as the “Internet” 2228. Local network 2222 and Internet 2228 both use electrical, electromagnetic, or optical signals that carry digital data streams. The signals through the various networks and the signals on network link 2220 and through communication interface 2218, which carry the digital data to and from computer system 2200, are example forms of transmission media.

Computer system 2200 can send messages and receive data, including program code, through the network(s), network link 2220, and communication interface 2218. In the Internet example, a server 2230 might transmit a requested code for an application program through the Internet 2228, ISP 2226, local network 2222, and communication interface 2218. The received code may be executed by processor 2204 as it is received, and/or stored in storage device 2210, or other non-volatile storage for later execution.

Operations of processes described herein can be performed in any suitable order unless otherwise indicated herein or otherwise clearly contradicted by context. Processes described herein (or variations and/or combinations thereof) may be performed under the control of one or more computer systems configured with executable instructions and may be implemented as code (e.g., executable instructions, one or more computer programs or one or more applications) executing collectively on one or more processors, by hardware or combinations thereof. The code may be stored on a computer-readable storage medium, for example, in the form of a computer program comprising a plurality of instructions executable by one or more processors. The computer-readable storage medium may be non-transitory. The code may also be provided carried by a transitory computer readable medium e.g., a transmission medium such as in the form of a signal transmitted over a network.

Conjunctive language, such as phrases of the form “at least one of A, B, and C,” or “at least one of A, B and C,” unless specifically stated otherwise or otherwise clearly contradicted by context, is otherwise understood with the context as used in general to present that an item, term, etc., may be either A or B or C, or any nonempty subset of the set of A and B and C. For instance, in the illustrative example of a set having three members, the conjunctive phrases “at least one of A, B, and C” and “at least one of A, B and C” refer to any of the following sets: {A}, {B}, {C}, {A, B}, {A, C}, {B, C}, {A, B, C}. Thus, such conjunctive language is not generally intended to imply that certain embodiments require at least one of A, at least one of B and at least one of C each to be present.

The use of examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

In the foregoing specification, embodiments of the invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

Further embodiments can be envisioned to one of ordinary skill in the art after reading this disclosure. In other embodiments, combinations or sub-combinations of the above-disclosed invention can be advantageously made. The example arrangements of components are shown for purposes of illustration and combinations, additions, re-arrangements, and the like are contemplated in alternative embodiments of the present invention. Thus, while the invention has been described with respect to exemplary embodiments, one skilled in the art will recognize that numerous modifications are possible.

For example, the processes described herein may be implemented using hardware components, software components, and/or any combination thereof. The specification and drawings are, accordingly, to be regarded in an illustrative rather than a restrictive sense. It will, however, be evident that various modifications and changes may be made thereunto without departing from the broader spirit and scope of the invention as set forth in the claims and that the invention is intended to cover all modifications and equivalents within the scope of the following claims.

All references, including publications, patent applications, and patents, cited herein are hereby incorporated by reference to the same extent as if each reference were individually and specifically indicated to be incorporated by reference and were set forth in its entirety herein.

What is claimed is:

1. A computer-implemented method for administering a treatment, comprising:
under the control of one or more computer systems configured with executable instructions:
determining a set of patient inputs;
determining a treatment;
estimating a circadian trajectory of the patient;
determining one or more circadian-mapping profiles;
determining, from the circadian trajectory and the one or more circadian-mapping profiles, a preferred treatment time period; and
administering the treatment in response to an alert that the preferred treatment time period is occurring or is to occur.

2. The computer-implemented method of claim 1, wherein the time period is one day.

3. The computer-implemented method of claim 1, wherein the time period is one minute.

4. The computer-implemented method of claim 1, wherein a length of the time period varies according to an uncertainty measure of the circadian trajectory, with the length being longer when the uncertainty measure is higher and the length being shorter when the uncertainty measure is lower.

5. The computer-implemented method of claim 1, wherein the treatment is the administration of a substance.

6. The computer-implemented method of claim 5, wherein the substance is one or more of a drug, nutrient, or medicament.

7. The computer-implemented method of claim 1, wherein the set of patient inputs comprises data derived from signals received of a patient from wearing a wearable data system.

8. The computer-implemented method of claim 1, wherein the circadian trajectory is derived by a scheduler using at least one biophysics model of a human circadian clock and at least one a statistical model of the human circadian clock.

9. The computer-implemented method of claim 1, wherein the preferred treatment time period is optimized based on associating a circadian time with a time for taking a drug for generating a raw model output.

10. The computer-implemented method of claim 1, further comprising presenting patient treatment outputs in a human-interpretable form.

11. The computer-implemented method of claim 1, further comprising:
connecting a model output to environmental controls to adjust an environment such that circadian-relevant behaviors are adjusted towards a target-constrained time.

12. The computer-implemented method of claim 11, wherein the treatment is administered at an infusion clinic.

13. The computer-implemented method of claim 11, wherein the treatment is a scheduled surgery.

14. The computer-implemented method of claim 1, further comprising filling in gaps in one or more of the models in missing data according to a ruleset.

15. The computer-implemented method of claim 1, further comprising providing a model that generates an optimal preferred treatment time period wherein the treatment is the administering of a drug and wherein the model associates a circadian time with a time for taking the drug, the model including rules representing how different molecules in the drug and body bind and interact with each other.

16. The computer-implemented method of claim 15, wherein the rules include variables representing molecular equations and parameters represent binding rates.

17. The computer-implemented method of claim 1, further comprising providing a model that generates an optimal time for taking a drug by associating a circadian time with a time for taking a drug for generating a raw model output and gating the triggering alerts based on logical gating in order to provide an optimal time for spacing treatments.

18. The computer-implemented method of claim 1, further comprising providing a model that generates an optimal time for taking a drug by associating a circadian time with a time for taking a drug for generating a raw model output, the model of the core circadian pacemaker can be connected to a model of the blood-brain barrier.

19. The computer-implemented method of claim 1, further comprising providing a model that generates an optimal time for taking a drug by associating a circadian time with a time for taking a drug for generating a raw model output, the model of the core circadian pacemaker can be connected to a model of the liver.

20. The computer-implemented method of claim 1, further comprising providing a mechanism converting the raw model output into a human-interpretable form, the method provides an optimal time for taking a drug, subject to a rule that a patient can take only one of these drugs in a particular day.

21. A non-transitory computer-readable storage medium storing instructions, which when executed by at least one processor of a computer system, causes the computer system to carry out the method of claim 1.

22. A computer system comprising:
one or more processors; and
a storage medium storing instructions, which when executed by the at least one processor, cause the system to implement the method of claim 1.
